(19) United States
(12) Patent Application Publication
(10) Pub. No.: US 2014/0364942 A1
(43) Pub. Date: Dec. 11, 2014

(54) SYSTEM AND METHOD FOR LOADING A STENT INTO A MEDICAL DELIVERY SYSTEM

(71) Applicants: Helmut Straubinger, Aschheim (DE); Johannes Jung, Pforzheim-Huchenfeld (DE); Arnulf Mayer, Bulach (CH)

(72) Inventors: Helmut Straubinger, Aschheim (DE); Johannes Jung, Pforzheim-Huchenfeld (DE); Arnulf Mayer, Bulach (CH)

(73) Assignee: JenaValve Technology GmbH, Munich (DE)

(21) Appl. No.: 14/346,143

(22) PCT Filed: Sep. 10, 2012
(86) PCT No.: PCT/EP2012/067617

§ 371 Date: Jul. 9, 2014

(30) Foreign Application Priority Data
Sep. 30, 2011 (EP) 11183541.9

(51) Int. Cl. A61F 2/95; A61F 2/24

(52) U.S. Cl. CPC A61F 2/95; A61F 2/2418; A61F 2002/9522
USPC 623/2.11

(57) ABSTRACT

The present disclosure relates to a device (100) for compressing and releasably connecting a stent (50), in particular the self-expanding stent (50) having a replacement placement heart valve (60) affixed thereto, with retaining means (70) of a delivery catheter system, in particular with retaining means (70) provided in or at a catheter tip (80, 80-1, 80-2) of a delivery catheter system. The loading system (100) comprises fixing means (10) for releasable fixing the stent (50) and centering means (30) connectable to the fixing means (10) for centering the stent (50) fixed to the fixing means (10). The fixing means (10) comprises a cup-shaped element (11) having a rim zone (12) formed inside the cup-shaped element (11) for clamping the stent (50). The centering means (30) comprises a frustoconical housing (31) having an open end (31.2) opposite to the fixing means (10). The housing (31) is configured to compress the stent (50) when the stent (50) is moved through the housing (31).

The present disclosure relates generally to a system and method for compressing and releasably connecting a stent with retaining means of a delivery catheter system. The invention may also be used to load a stent, in particular a stent having a prosthetic heart valve affixed thereto (hereinafter also referred as “prosthetic cardiac valve assembly”) or other non-valvular prosthetic frames onto a minimally invasive delivery system, such as a delivery catheter, for example.

Medical technology has long since endeavored to correct valvular defects such as, for example, aortic valve insufficiencies or aortic valve stenosis, by means of non-surgical, transarterial access; i.e. without requiring open heart surgery, with implantation by way of catheter. In the process, various different stent systems with various different advantages and disadvantages have been proposed, some which can also be inserted transarterially into the body of a patient via a catheter delivery system.

The terms “aortic valve stenosis and/or aortic valve insufficiency” as used herein generally refer to a congenital or acquired dysfunction of one or more cardiac valves. Such valvular disorders can affect any of the four cardiac valves, whereby the valves in the left ventricle or left chamber (aortic and mitral valve) are typically more affected than those on the right side of the heart (pulmonary and tricuspid valve). The dysfunction can be a constriction (stenosis), an incompetence (insufficiency) or a combination of the two (combined vitium).

Minimally invasive forms of treatment have recently been developed which are in particular characterized by allowing the procedure to be performed under local anesthesia. One approach provides for using a catheter system to implant an expandable stent, to which a collapsible prosthetic heart valve has been affixed, into a human body. Such an expandable prosthetic heart valve can be guided via a delivery or catheter system to the implantation site within the heart through an inguinal artery or vein. After reaching the implantation site, the stent can then be unfolded. After unfolding, the prosthetic heart valve can be anchored in the respective blood vessel at least in an area close to the heart, for example with the aid of anchoring hooks. The actual prosthetic heart valve is usually positioned in the inflow area of the stent.

For example, document WO 2004/019825 A1 describes a heart valve stent for a heart valve prosthesis having a stent a prosthetic heart valve affixed to the stent. This heart valve prosthesis can be introduced into the site of implantation in the patient's heart via a medical delivery system to treat an aortic valve stenosis and/or aortic valve insufficiency in a minimally invasive manner.

Known conventional systems for implanting a prosthetic heart valve introduce an expandable stent system transarterially/transfemorally or transapically into the body of the patient using a medical delivery system. This type of stent system consists for example of an expandable anchoring support (hereinafter also referred to as “cardiac valve stent” or simply “stent”), to which the actual prosthetic heart valve is affixed or can be affixed, preferably at the end region nearest the heart (inflow end).

The explanations disclosed herein with respect to a “stent system” are also applicable to a “stent”.

The term “medical delivery system” as used herein generally refers to a medical system with which a stent system can be advanced in minimally invasive fashion to the site of implantation in the patient's heart, for example to treat an aortic valve stenosis and/or aortic valve insufficiency. In the present context, “minimally invasive” means a heart-lung machine is not needed when performing the procedure on the anaesthetized patient such that not only can the medical procedure be performed at reasonable cost, but there is also less physical and psychological strain on the patient.

A medical delivery system usually comprises a catheter system by means of which a stent, as needed with a prosthetic heart valve affixed thereto in folded state, can be introduced into the patient's body in its folded state. For example, the medical delivery system can exhibit a catheter tip having at least one manipulatable receiving area at a distal end section of the catheter system; i.e. closest to the heart. It is moreover conceivable for the medical delivery system to exhibit a handle at the proximal end section of the catheter system; i.e. at the end section of the catheter system farthest from the heart and the catheter tip, with which the at least one receiving area of the caterer tip can be appropriately manipulated such that the expandable stent accommodated in the catheter tip, as needed with a prosthetic heart valve affixed thereto, can be incrementally released from the catheter tip according to a predefined or predefinable sequence of events.

In this disclosure, the expression “catheter system” means a system that can be inserted into a body cavity, duct or vessel. A catheter system thereby allows access by surgical instruments. The process of inserting a catheter system is catheterisation. In most uses a catheter system is a thin, flexible tube: a “soft” catheter system; in some uses, it is a larger, solid tube: a “hard” catheter system. A catheter system for a minimally invasive implantation and transvascular implantation of prosthetic heart valves is described, for example, in document WO 2006/076890 A1.

To introduce the stent system, the stent together with the prosthetic heart valve affixed as needed thereto, is loaded into the tip of the medical delivery system's catheter. In order to do so, the stent, as needed with the prosthetic heart valve affixed thereto, needs to exhibit a first predefinable shape in which the stent or the stent and the prosthetic heart valve affixed thereto is/are in a compressed or folded state. In its first predefined state, the stent, as needed with the prosthetic heart valve affixed thereto, exhibits a diameter which is essentially determined by the diameter of the catheter tip of the medical delivery system.

For the majority of patients undergoing treatment, it is preferable for the stent, as needed with the prosthetic heart valve affixed thereto, to have an outer diameter of approximately 7.0 mm to approximately 5.0 mm in its first shape so that the stent system can be introduced with a 23F delivery system (given an external diameter of 7.0 mm) or with a 17F delivery system (given an external diameter of 5.0 mm). In some cases, however, a delivery system having a larger outside diameter (up to 32 French) is necessary for introducing, for example, a stent system, i.e. a stent with a heart valve prosthesis affixed thereto.

After the stent system has been released from the catheter tip, in the implanted state respectively, the stent system exhibits a second predefined shape in which the stent or the stent and the prosthetic heart valve affixed thereto is/are in an expanded state. Depending on the patient being treated, it is preferable for the stent to exhibit a diameter of between 19.0 mm and 27.0 mm in its second shape and implanted state.

Thus, the first shape transitions to the second shape by a cross-sectional widening, wherein the stent stretches radially and presses against the vascular wall of a blood vessel near the heart and thus fixes a prosthetic heart valve affixed as needed to the stent at the site of implantation. The cross-sectional widening can be effected by a balloon system when the stent is implanted with the help of a so called balloon catheter system.

On the other hand, it is also known from medical technology to construct the stent from a superelastic shape memory material which is designed such that the stent can transform from a temporary shape into a permanent shape under the influence of an external stimulus. The temporary shape thereby corresponds to the stent's first shape when the stent, as needed with the prosthetic heart valve affixed thereto, is in its folded state. The permanent shape corresponds to the stent's second shape when in its expanded state. An example of a suitable shape memory material would be Nitinol, e.g., an equiatomic alloy of nickel and titanium.

Turning out to be disadvantageous with conventional systems for implanting a prosthetic heart valve as known to date, however, has been that not only the actual implantation of the stent, as needed with the prosthetic heart valve affixed thereto, but also the preparation needed for the implant procedure is relatively complicated, difficult and laborious. Apart from the complicated implanting of the stent, as needed with a prosthetic heart valve affixed thereto, to replace an insufficient native heart valve, for example, there is also the fundamental problem of the stent and/or the stent and a prosthetic heart valve affixed thereto being damaged when the stent, as needed with a prosthetic heart valve affixed thereto, is loaded into the tip of the catheter of the medical delivery system in preparation for the surgery. In particular with self-expanding stent systems, the stent, as needed with a prosthetic heart valve affixed thereto, has to be compressed so that it will then be in its first shape and be able to be introduced into the tip of the catheter of a medical delivery system. This subjects the stent to considerable compressive forces in order to overcome the self-expanding stent structure's expansion forces and achieve the desired reduction in cross-section.

While it is known to fabricate a stent in an initial fabrication orientation, in which the stent has an initial fabrication diameter and an initial fabrication length, such that the stent may be easily loaded onto a delivery catheter, such conventional solutions are not readily adaptable for use with prosthetic valve assemblies, i.e. stents having a prosthetic heart valve attached thereto.

For example, the document U.S. Pat. No. 5,911,752 A discloses a system for loading expandable vascular grafts or stents for use within body passageways onto a delivery catheter. More particularly, expandable stents for maintaining the patency of blood vessels are taken into account in this prior art. According to the conventional approach, the stents are already loaded onto a delivery catheter at the manufacturing facility prior to shipping. Alternatively, the stents may be shipped in an at least partially collapsed orientation. In such a partially collapsed orientation, the outer diameter of the stent is sufficiently small to allow physicians to load the stent onto a delivery catheter.

The present system as known, for example, from document U.S. Pat. No. 5,911,752 A, however, is not readily adaptable for use with prosthetic valve assemblies, i.e. stents having a prosthetic heart valve attached thereto. Contrary to stents for maintaining the patency of blood vessels, prosthetic valve assemblies are normally shipped in their expanded orientation. Then, the stent together with the heart valve prosthesis affixed thereto needs to be compressed and inserted into a catheter tip of a catheter system shortly before surgery. Accordingly, there is a need for a system which is adapted to load a prosthetic heart valve assembly onto a delivery catheter. In particular, such a system must be easily operatable by physicians.

Moreover, present systems for loading expandable stent systems onto a delivery catheter are not readily adaptable for use with prosthetic valve assemblies, because such systems are many times only configured for stents without any valve prosthesis attached thereto. Hence, such conventional systems prone to damaging the valve portion of the prosthetic valve assembly when used in connection with a prosthetic valve assembly.

Similar circumstances however also apply to stent systems which are implanted using balloon catheter systems.

Accordingly, a need exists for a suitable system and method of loading a prosthetic valve onto a delivery system, such as a delivery catheter, for example.

In conjunction hereto, often likewise regarded as problematic is that when preparing for the implant procedure, the stent, as needed with a prosthetic heart valve affixed thereto, can often only be loaded into the tip of the catheter of a medical delivery system by an experienced perfusionist or by product specialists so as to avoid damaging the stent system and so that the stent system can be properly transformed into its defined first shape.

Without special compressing mechanisms or loading systems, the known systems are thus coupled with the fundamental risk of damage to the stent system or it not properly being transformed into its defined first shape, for example due to an oversight on the part of the perfusionist or product specialist or some other incident occurring during the compressing of the stent system. Damage which occurs when compressing the stent system or when loading the compressed stent system into the catheter tip of the medical delivery system are often not noted until the actual implant procedure is underway, for example when the positioning and/or fixing of the prosthetic heart valve at the site of implantation at the heart by means of the stent is imprecise, when the stent will not properly expand at the implantation site in the heart, or when it is for example determined that the implanted prosthetic heart valve cannot or not adequately enough assume the function of the native heart valve to be replaced.

Furthermore, present systems for loading expandable stent systems onto a delivery catheter are not readily adaptable for precisely and properly connecting the stent with corresponding retaining means which are often provided in or at the catheter tip of the delivery catheter. On the other hand, however, a stent not precisely connected with corresponding retaining means of the delivery catheter is not releasable in a predefined sequence of events in the implantation site in the patient's body.

On the basis of the problems outlined above, the present disclosure relates to a system as well as a method with which at least parts of a stent, as needed with a prosthetic heart valve affixed thereto, can be readily compressed to a desired diameter while at the same time the stent can be releasably connected with retaining means of a delivery catheter system, in particular without the risk of the stent and/or the stent and a prosthetic heart valve affixed thereto being damaged.

Embodiments of the present disclosure may provide a simplified method for loading a stent, as needed with a prosthetic heart valve affixed thereto, into the catheter tip of a medical delivery system, in particular wherein the proper loading of the stent into the tip of the catheter and the proper connecting of the stent with corresponding retaining means of a delivery catheter system and the proper connecting of the stent with corresponding retaining means of a delivery catheter system no longer depends to a significant extent on the finesse and experience of the given perfusionist or product specialist.

Preferably, the system and method provide a tool for simplifying the process of loading of a transapically or transluminally implantable stent, for example, a prosthetic valve, onto a delivery catheter, or other delivery system, for a minimally invasive implantation of the prosthesis into the vasculature at a location remote from the implantation site. Preferred embodiments of the present invention preferably are used with a self-expanding prosthesis, but may also be useful in connection with balloon-expandable or other mechanically-expanded prostheses. Desirably, preferred embodiments of the present invention permit the reduction of an external dimension of a compressible valve prosthesis without damaging the valve.

A preferred embodiment is a system for compressing and releasably connecting stent with retaining means of a delivery catheter system, wherein the system comprises fixing means for releasable fixing the stent and centering means connectable to the fixing means for centering the stent fixed to the fixing means. The fixing means may comprise a cup-shaped element having a rim zone formed inside the cup-shaped element for clamping the stent. The centering means preferably comprises a frustoconical housing having an open end opposite to the fixing means. The housing is configured to compress the stent when the stent is moved through the housing.

Embodiments of the present disclosure allow for compressing at least parts of the stent, as needed with a prosthetic heart valve affixed thereto, to a desired diameter. The term “desired diameter” means a diameter of the stent which allows a proper loading of the stent into the tip of a catheter.

For this purpose, the centering means having the frustoconical housing is provided. The frustoconical housing includes a tapered surface and is configured to reduce the external dimension of at least a portion of the stent when the stent is moved along this tapered surface. On the other hand, the frustoconical housing is adapted for centering the stent, in particular in relation to retaining means of a delivery catheter system, for example retaining means provided in or at a catheter tip of the delivery catheter system.

Although not mandatory, the rim zone formed inside the cup-shaped element of the fixing means may have a side surface extending substantially parallel to the longitudinal direction of the cup-shaped element and an upper surface extending perpendicular thereto. With such a rim zone, clamping means associated to the cup-shaped element of the fixing means are provided for releasable fixing the stent prior to centering the stent by using the centering means.

For providing the rim zone inside the cup-shaped element, the fixing means may comprise an annular element having a substantially rectangular cross section, wherein this annular element is accommodated or can be received in the cup-shaped element. Preferably, the inner diameter of the annular element is smaller than the outer diameter of a stent to be fixed with the fixing means.

In some embodiments of the system, an axially arranged cylindrical recessed portion may be formed in the cup-shaped element of the fixing means, thereby defining an inner shell and a bottom surface of the cup-shaped element. Then, the already mentioned annular element may be accommodated in the recessed portion, whereby at least parts of the annular element may be connected to the inner shell and the bottom surface of the cup-shaped element. Preferably, an opening and, in particular, a circular opening may be provided in the bottom surface of the cup-shaped element, wherein the diameter of this opening is smaller then the inner diameter of the annular element.

In some embodiments of the system, the cup-shaped element and the annular element are integrally formed as one piece, preferably from a plastic material.

Although not mandatory, the housing of the centering means may comprise a first cylindrical end region and an opposite second end region, wherein the first cylindrical end region can be at least partially received in the cup-shaped element of the fixing means. Preferably, the first cylindrical end region has a diameter which is larger then the inner diameter of the cup-shaped element at a region of the cup-shaped element where the rim zone is formed.

In some embodiments of the system an annular element having a substantially rectangular cross section is accommodated in the cup-shaped element of the fixing means such as to form the already mentioned rim zone inside the cup-shaped element. In this embodiment, the first cylindrical end region of the housing of the centering means may have a diameter which is larger then the inner diameter of the annular element.

The housing of the centering means may comprise a first cylindrical end region connectable to the fixing means and an opposite second end region, wherein an axially arranged frustoconical through-hole is formed in the housing of the centering means. The frustoconical through-hole preferably tapers toward the second end region of the housing. In this embodiment, the diameter of the frustoconical through-hole at the first end region of the housing is preferably smaller then the inner diameter of the cup-shaped element at a region where the rim zone is formed inside the cup-shaped element.

Furthermore, an annular element having a substantially rectangular cross section may be accommodated in the cup-shaped element of the fixing means such as to form the rim zone inside the cup-shaped element, wherein the diameter of the frustoconical through-hole at the first end region of the housing of the centering means is preferably smaller then the inner diameter of the annular element.

In one possible realization of the system, the diameter of the frustoconical through-hole at the first end region of the housing of the centering means is selected such that—when the centering means is connected to the fixing means—an annular groove is formed inside the system to receive parts of the stent and in particular parts of the replacement heart valve affixed to the stent. The annular groove is at least partly limited by the side surface of the rim zone formed in the cup-shaped element, the bottom surface of the cup-shaped element and a base of the first cylindrical end region of the housing.

For achieving a releasable connection between the fixing means and the centering means, a preferred realization of the system provides for the fixing means to comprise at least one locking element, wherein the centering means comprises at least one locking element configured complementary to the locking element of the fixing means. It is hereby possible to releasable lock the fixing means to the centering means in the assembled state of the system.

One possible realization of the locking element provides for the locking element to comprise an L-shaped slot, wherein the locking element configured correspondingly complementary thereto is preferably formed as a projecting pin.

A preferred method includes providing a system according to the present invention, inserting a stent into the cup-shaped element of the fixing means of the inventive system, and joining the centering means of the inventive system to the fixing means to center the stent inserted into the cup-shaped element.

Embodiments of the present disclosure allow for loading prosthetic valve assemblies, i.e. stents having a prosthetic heart valve attached thereto, into a catheter tip of a catheter system shortly before surgery. In particular, the systems of the present disclosure are easily operatable by physicians. Furthermore, the risk of damaging the valve portion of the prosthetic valve assembly when loaded onto the delivery catheter is considerably reduced.

These and other features, aspects and advantages of the present invention are described in greater detail below in connection with drawings of a preferred system and method, which is intended to illustrate, but not to limit the present invention.

Shown are:

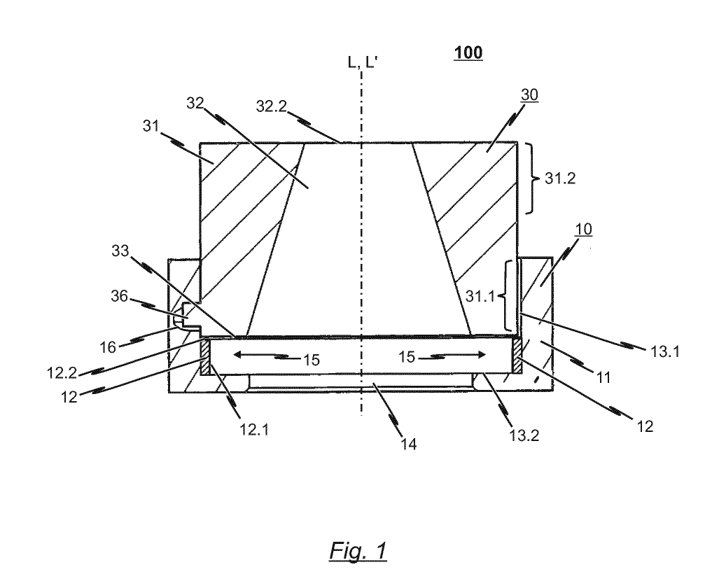

FIG. 1 a cross-sectional view of an exemplary embodiment of the inventive system for compressing and releasably connecting a stent with retaining means of a delivery catheter system, wherein the system comprises fixing means and centering means connected to the fixing means;

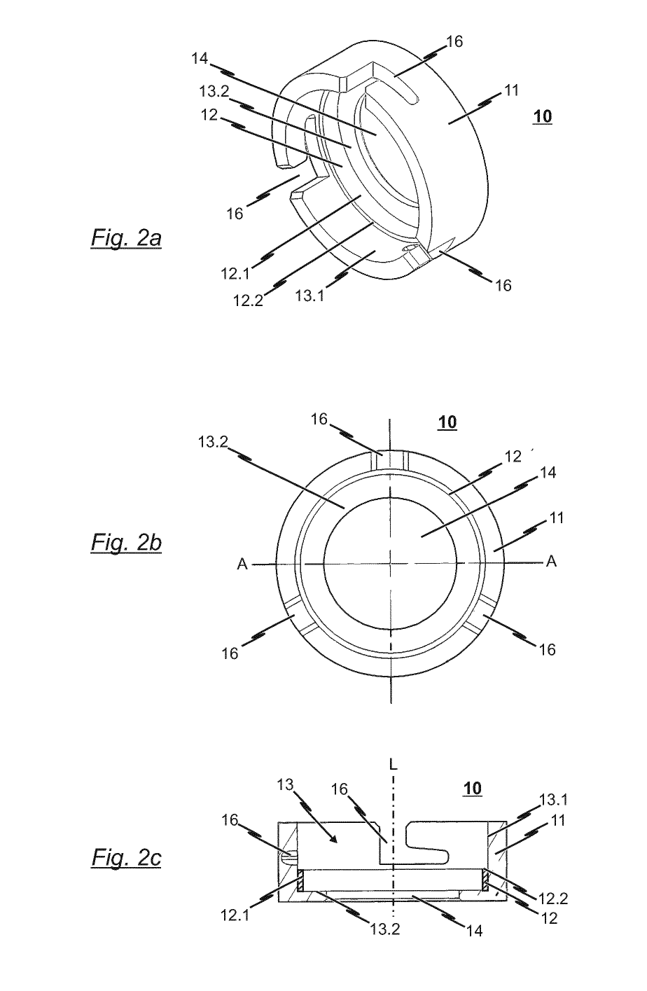

FIG. 2a a perspective view of the fixing means utilized in the exemplary embodiment of the inventive system depicted in FIG. 1;

FIG. 2b a top view of the fixing means of FIG. 2a;

FIG. 2c a cross-sectional view of the fixing means of FIG. 2a, taken a long view line A-A of FIG. 2b;

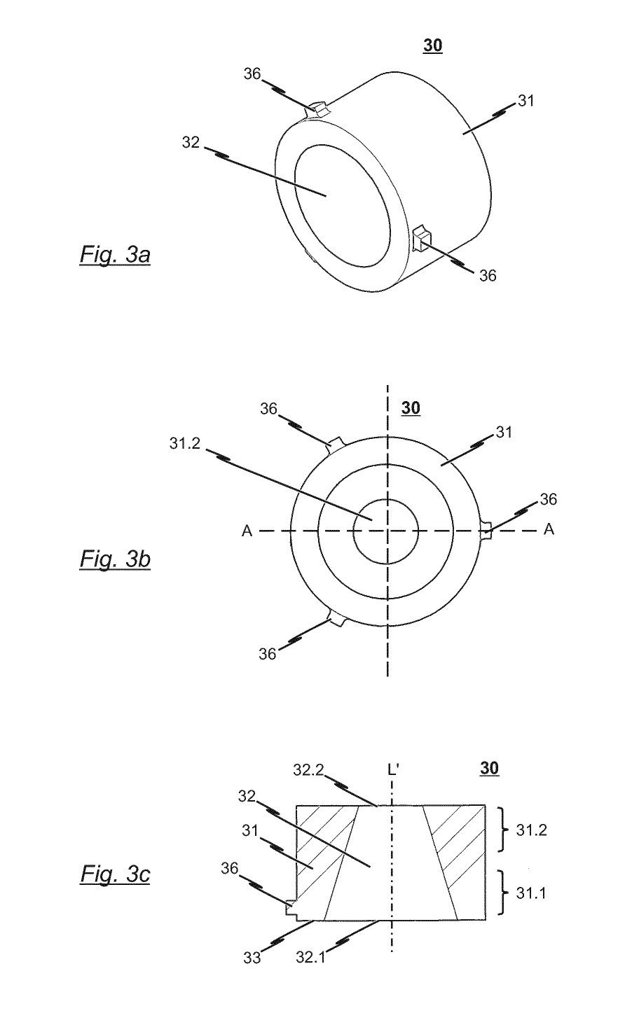

FIG. 3a a perspective view of the centering means utilized in the exemplary embodiment of the inventive system depicted in FIG. 1;

FIG. 3b a top view of the centering means of FIG. 3a;

FIG. 3c a cross-sectional view of the centering means of FIG. 3a, taken a long view line A-A of FIG. 3b;

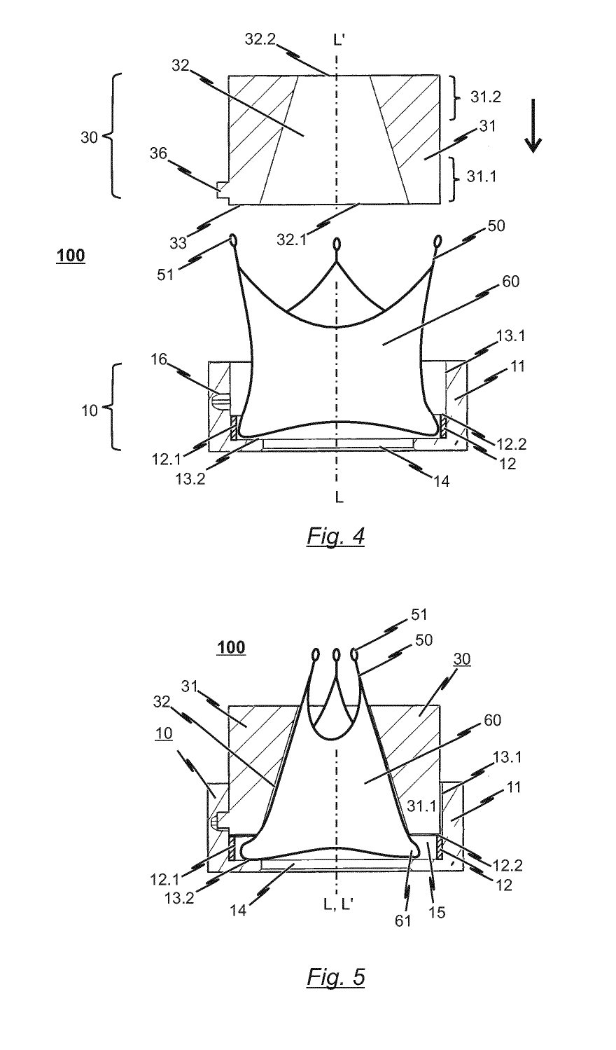

FIG. 4 a cross-sectional view of the exemplary embodiment of the inventive system according to FIG. 1 in its unassembled state, wherein a self-expanding stent having a replacement heart valve affixed thereto is connected to the fixing means and wherein the centering means is not connected with the fixing means;

FIG. 5 a cross-sectional view of the exemplary embodiment of the inventive system according to FIG. 1 in its assembled state, wherein a self-expanding stent having a replacement heart valve affixed thereto is connected to the fixing means and wherein the centering means is connected with the fixing means for centering and partly compressing the stent connected to the fixing means;

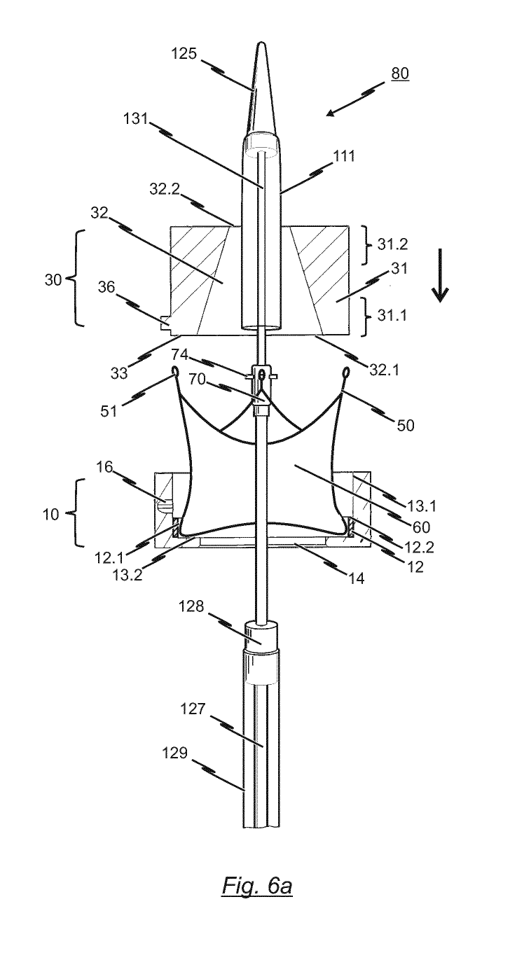

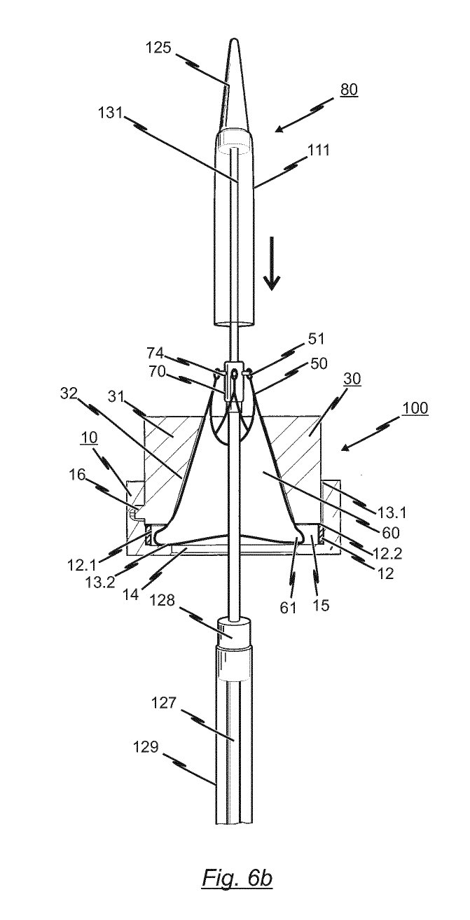

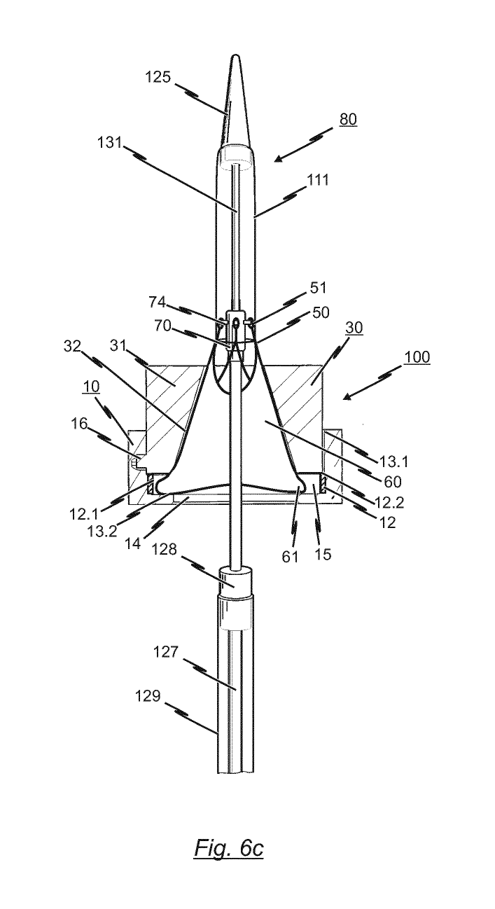

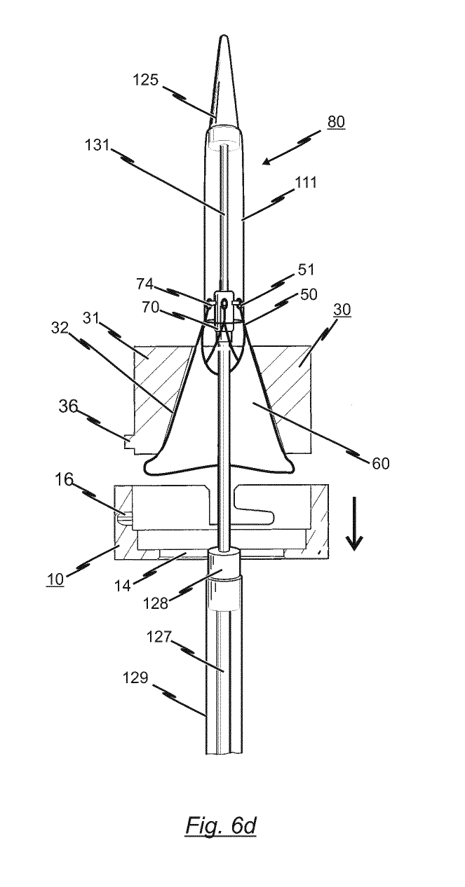

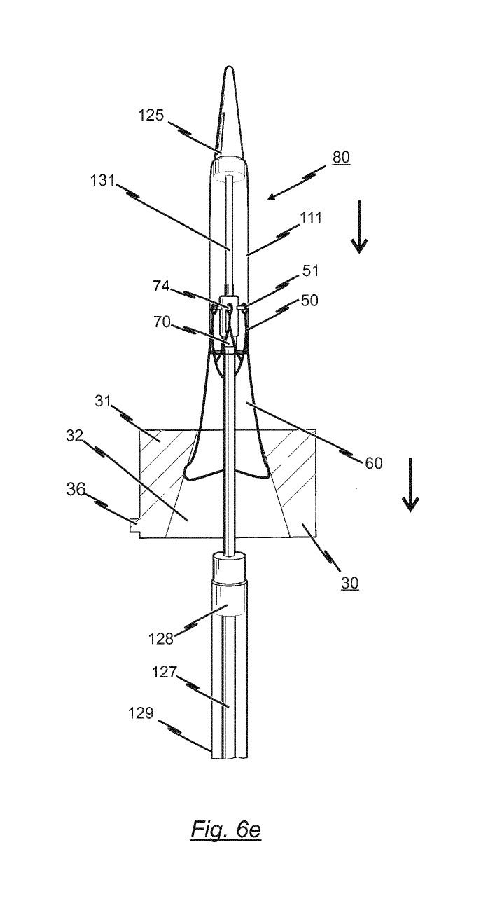

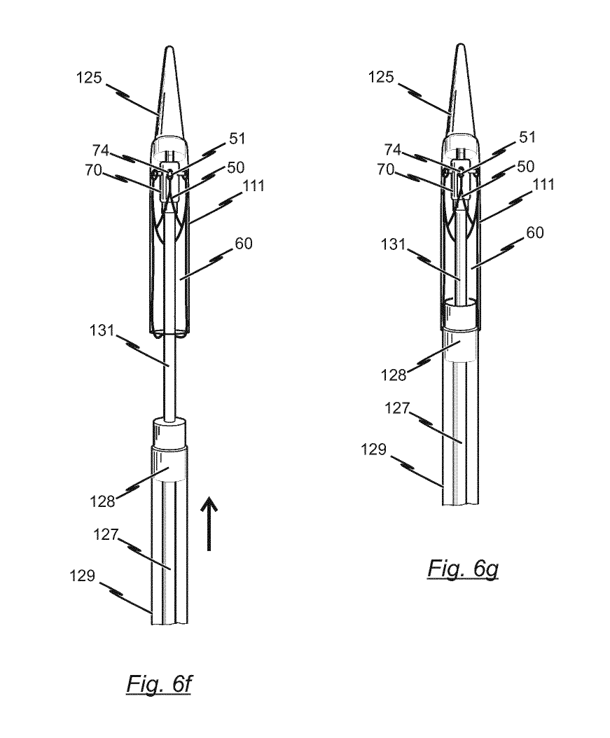

FIG. 6a-g a method of loading a valve prosthesis into the catheter tip of a delivery catheter system;

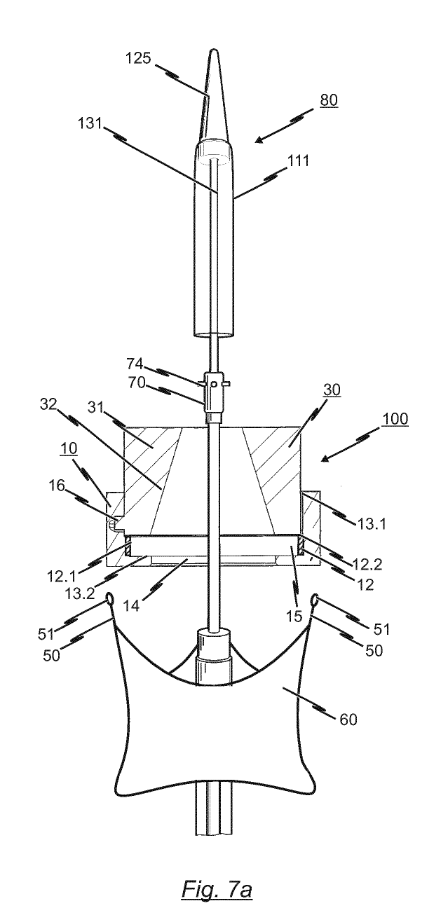

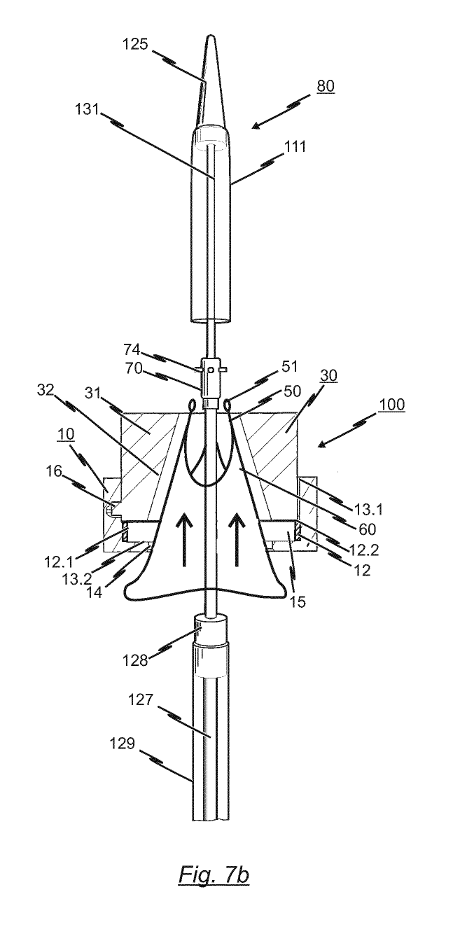

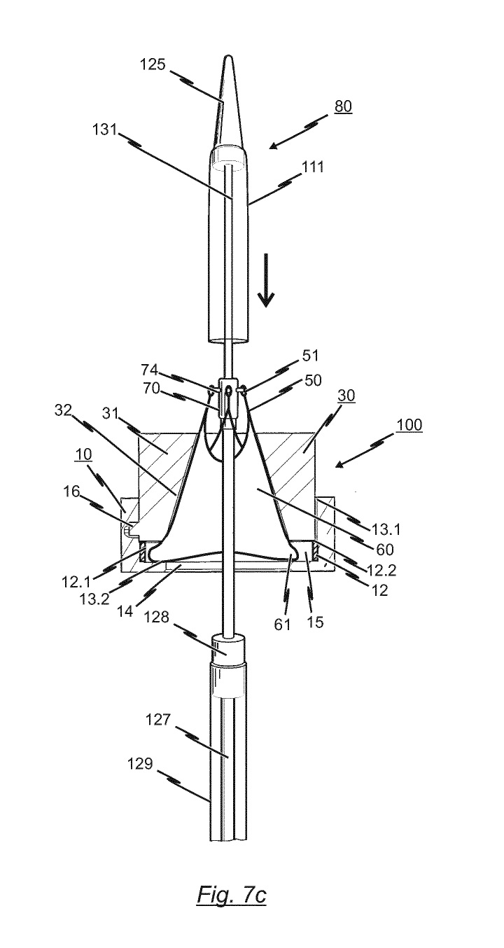

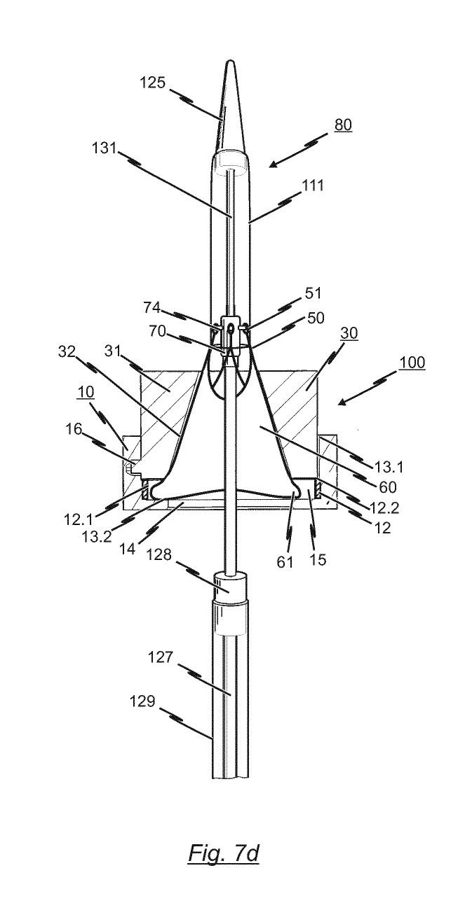

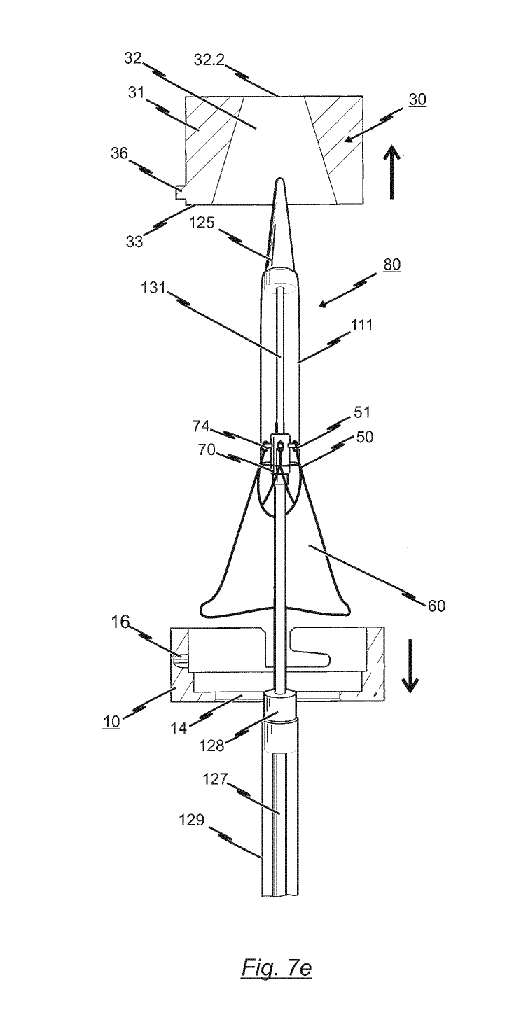

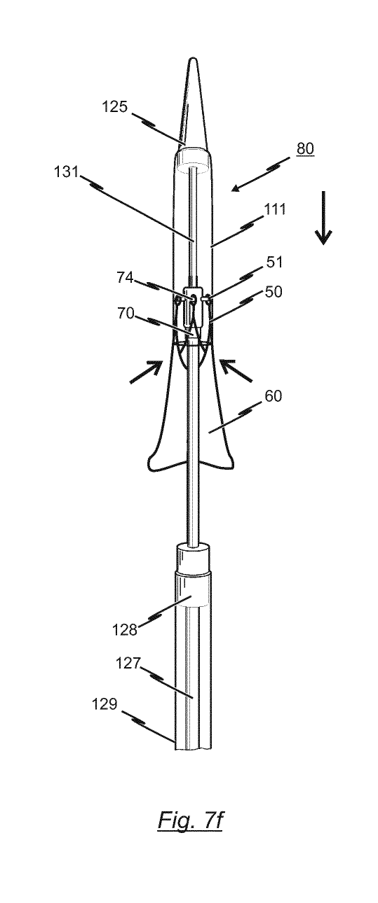

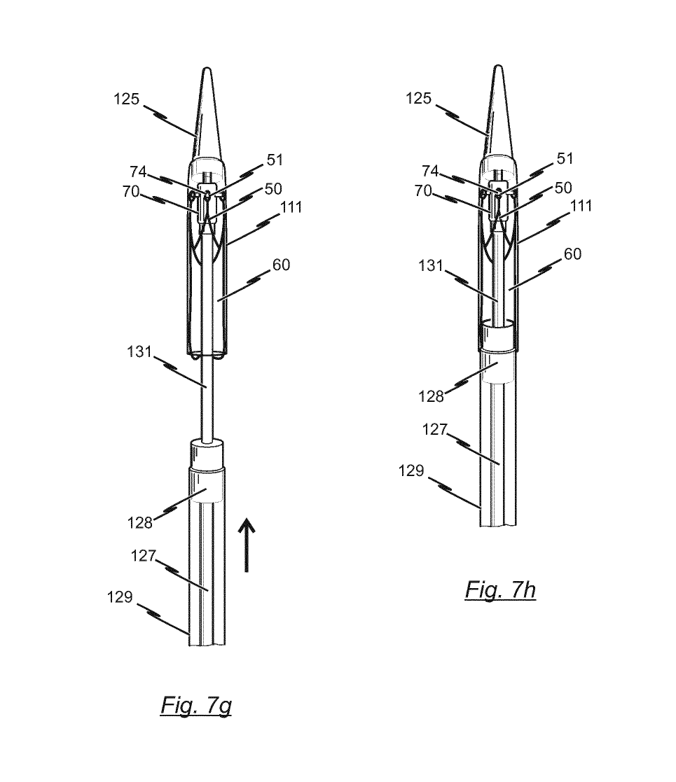

FIG. 7a-h another method of loading a valve prosthesis into the catheter tip of a delivery catheter system;

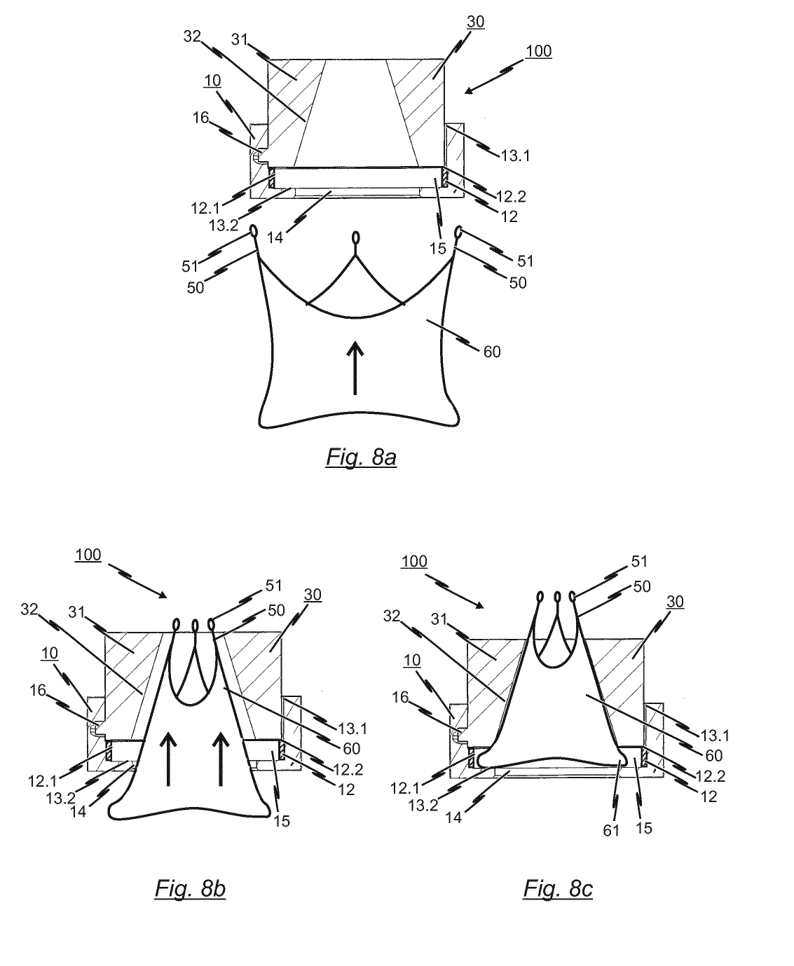

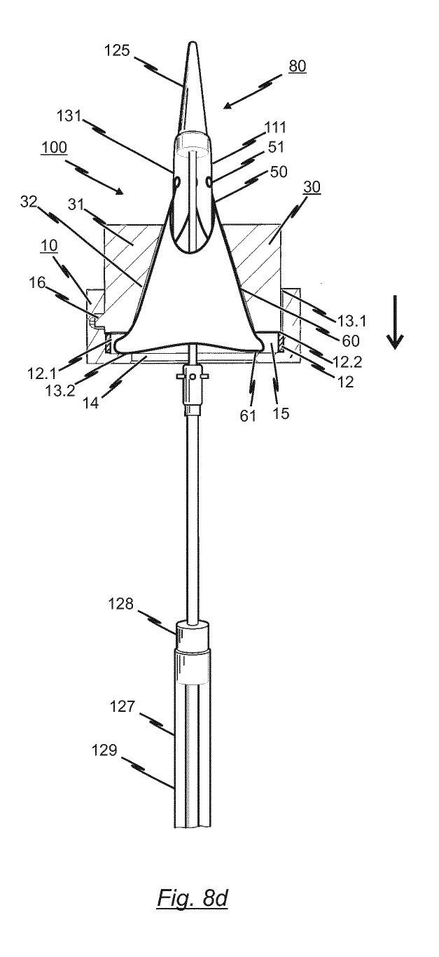

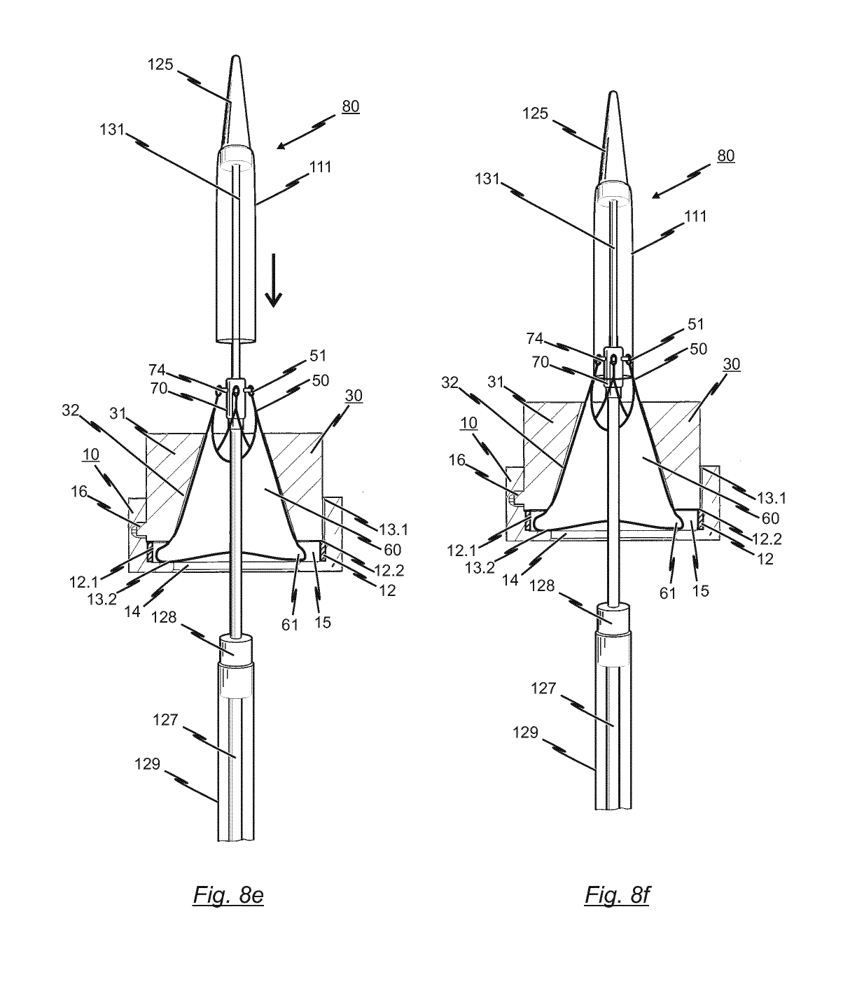

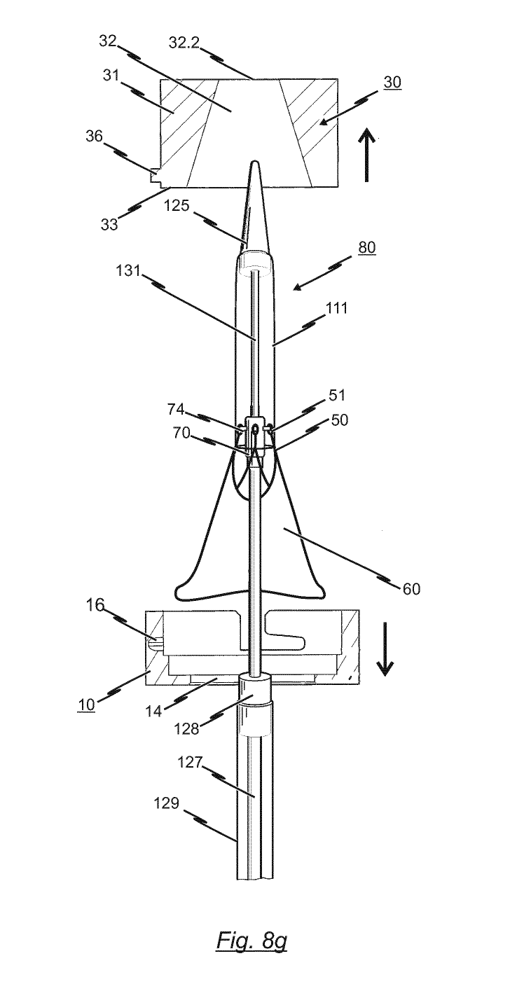

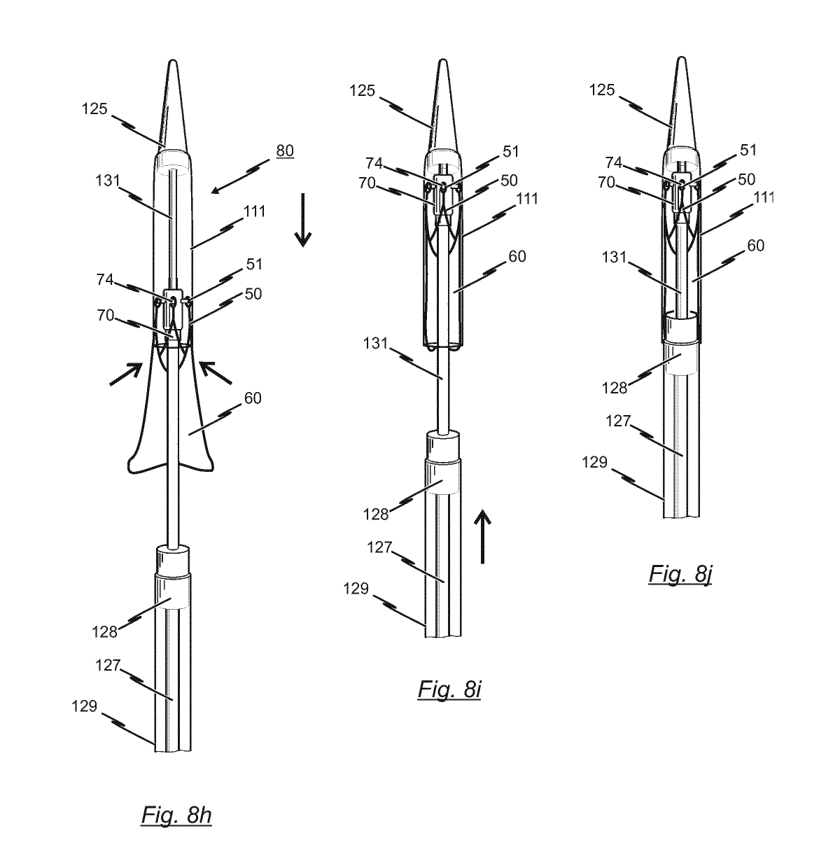

FIG. 8a-j another method of loading a valve prosthesis into the catheter tip of a delivery catheter system;

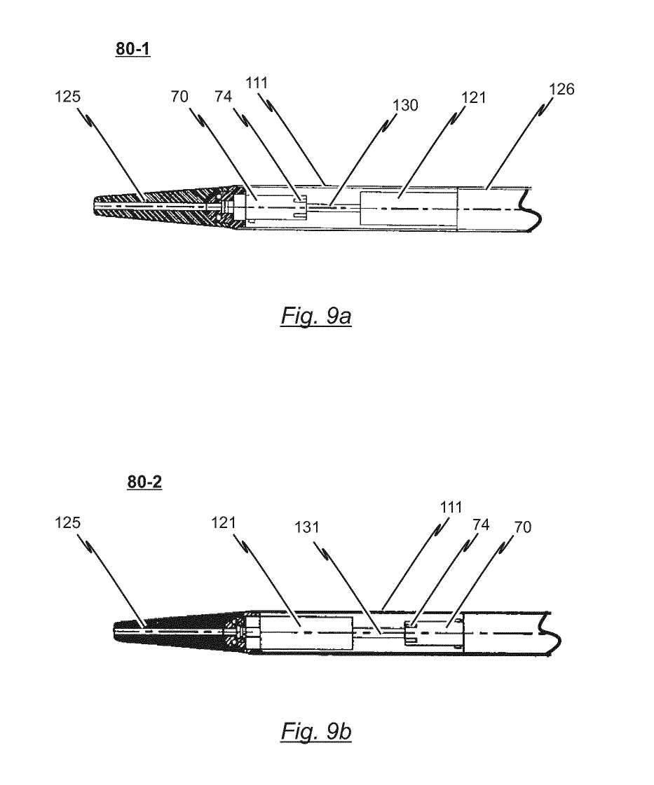

FIG. 9a a side view of an exemplary embodiment of a catheter tip of a medical delivery system for transapically introducing a stent;

FIG. 9b a side view of an exemplary embodiment of a catheter tip of a medical delivery system for transfemorally/transarterially introducing a stent;

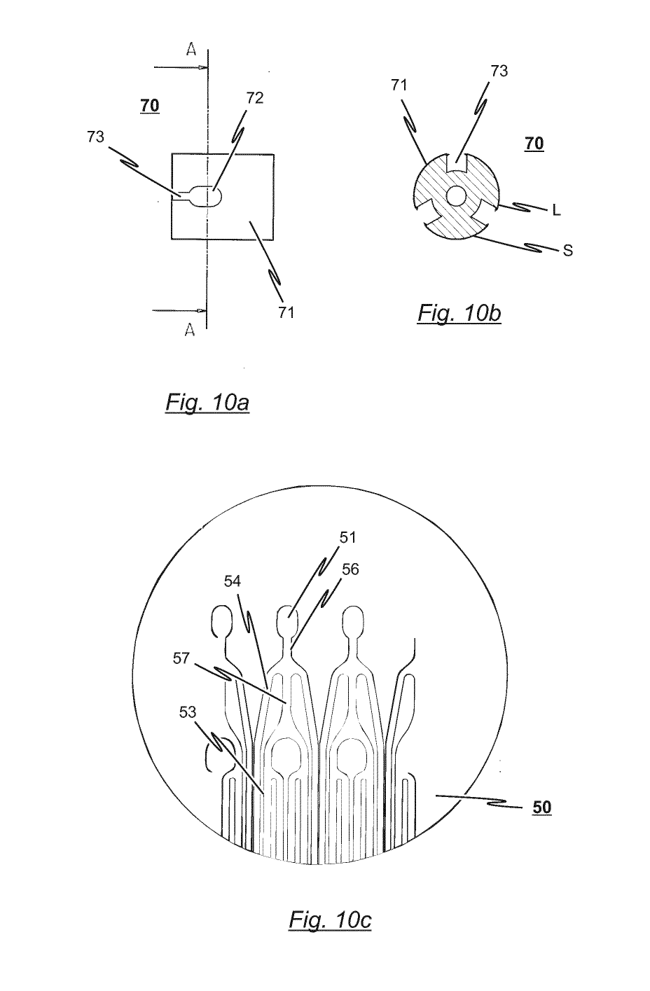

FIG. 10a a side view of an embodiment of retaining means disposed in a catheter tip of a medical delivery system for transapically or transfemorally/transarterially introducing a stent;

FIG. 10b a view in cross-section of the retaining means illustrated in FIG. 10a, seen along line A-A indicated in FIG. 10a; and

FIG. 10c a plan view of the proximal retaining region of a stent having retaining elements, which can be retained by means of the retaining means illustrated in FIG. 10a.

Although there has been considerable development and refinement of vascular stent concepts in relationship to the coronary vasculature for the treatment of myocardial infarction and angina, these concepts do not necessarily translate to prosthetic structures involving larger sections of vasculature, and more specifically, implants incorporating prosthetic valve for minimally invasive delivery from peripheral access sides of the body. For example, although a small delivery profile, or small cross-sectional configuration, is desirable for both coronary stents and prosthesis valve, the expanded size and the implantation location of prosthesis valve may introduce difficulties into the loading of a prosthesis onto a catheter or other minimally invasive delivery system.

An exemplary embodiment of a catheter tip 80-1 of a medical delivery system, for example a delivery catheter, for transapically introducing an expanded stent into the body of a patient will be described below referencing FIG. 9a. The inventive system described below for example with reference to FIGS. 1 to 5 is suited to loading a stent 50 into the catheter tip 80-1 depicted in FIG. 9a; although the disclosure is in no way limited to the use of the system in combination with the catheter tip 80-1 shown in FIG. 9a. Rather, the following description only serves to present an example of the design of a catheter tip 80-1 of a medical delivery system designed for a transapical approach, whereby the system aids in loading a stent 50, as needed with a prosthetic heart valve 60 affixed thereto, into said catheter tip 80-1.

The catheter tip 80-1 depicted in FIG. 9a is part of a medical delivery system, not further shown in FIG. 9a, which is suited for a transapical approach to a heart valve to be treated, such as for example an aortic valve.

The medical delivery system enables an expandable heart valve stent 50 to be implanted transapically in a patient's body; i.e. advanced from the apex of the heart. To this end, the delivery system comprises a catheter system (not shown in FIG. 7a) by means of which the stent 50 (likewise not depicted in FIG. 9a) can be positioned in its folded or compressed state in the patient's body.

The catheter tip 80-1 shown in FIG. 9a is disposed at the distal end section of the catheter system where the stent 50 to be implanted in the patient's body can be accommodated. A handle (not shown in FIG. 9a) can be provided at the proximal end section of the catheter system with which the catheter tip 80-1 can be manipulated.

In detail, the catheter tip 80-1 of the medical delivery system designed for transapical approach may comprise a stent holder 70 by means of which an end section of a stent 50 to be implanted into the body of the patient can be releasably fixed to the catheter tip 80-1. For this purpose, the end section of the stent 50 is provided with at least one retaining element 51, for example in the form of a projecting element.

The catheter tip 80-1 further comprises receiving means for receiving the stent 50 in its compressed state. Specifically, the receiving means for the catheter tip 80-1 exemplarily depicted in FIG. 9a may consist of a first receiving area 111 and a second receiving area 128.

As FIG. 9a indicates, the exemplary medical delivery system designed for a transapical approach provides for the first receiving area 111 of catheter tip 80-1 to be configured as a stent sheath connected to the distal end tip 125 of catheter tip 80-1 with its opening pointing toward the proximal end section 126 of catheter tip 80-1. The first receiving area 111 configured as a stent sheath forms the outer lateral surface of the catheter tip 80-1 when the latter—as shown in FIG. 9a—is in its closed state.

In the catheter tip 80-1 of the delivery system designed for a transapical approach, the second receiving area 128 of catheter tip 80-1 may be configured as a stent funnel with its opening pointing toward the distal end tip 125 of catheter tip 80-1. The end section opposite to the at least one retaining element 51 of a stent 50 to be implanted (not shown in FIG. 9a) can for example be received within the second receiving area 128 configured as a stent funnel when the stent 50 is in its compressed state. This end section, i.e. the end section opposite to the at least one retaining element 51 of a stent 50, is also referred herein as “proximal end section”.

For example, it is conceivable for the proximal end section of stent 50 to comprise retaining arches to which a prosthetic heart valve is affixed as needed. In such a case, the retaining arches of stent 50, and the prosthetic heart valve affixed as needed to the retaining arches, are accommodated within the second receiving area 128 of catheter tip 80-1 configured as a stent funnel.

In the closed state of catheter tip 80-1 (cf. FIG. 9a), the second receiving area 128 configured as stent funnel is telescopically received by the first receiving area 111 configured as stent sheath, whereby positioning arches of the stent 50 can for example be arranged between the outer lateral surface of the stent funnel and the inner lateral surface of the stent sheath when a corresponding heart valve stent 50 is accommodated in the catheter tip 80-1.

In the catheter tip 80-1 of a medical delivery system designed for a transapical approach as depicted in FIG. 9a, the second receiving area 128 of the catheter tip 80-1 may be—as noted above—configured as a stent funnel in the form of a tubular or sleeve-like element. The stent funnel (second receiving area 128) can be connected to actuating means of a handle via force transfer means (not explicitly shown in FIG. 9a) so that pulling or pushing forces can be transferred to the second receiving area 128 of the catheter tip 80-1 upon the actuating of the actuating means. In this way, the second receiving area 128 of the catheter tip 80-1 configured as stent funnel can be displaced in the longitudinal direction of the catheter tip 80-1 relative the stent holder 70 on the one hand and relative the first receiving area 111 configured as a stent sheath on the other hand.

As indicated above, it is possible for the first receiving area 111 of the catheter tip 80-1 of the medical delivery system designed for a transapical approach to be configured as a stent sheath, for example in the form of an elongated tube. The second receiving area 128 may be configured as a stent funnel, likewise for example in the form of an elongated tube. The inner diameter of the tubular or sleeve-shaped first receiving area 111 should thereby be selected to be larger than the outer diameter of the likewise tubular or sleeve-shaped second receiving area 128 such that the second receiving area 128 can be telescopically received inside the first receiving area 111.

The stent holder 70 of the catheter tip 80-1 for a medical delivery system designed for a transapical approach as depicted in FIG. 9a may be configured as a cylindrical element furnished with appropriate retaining means 74. The retaining means 74 serve to create a releasable connection to a retaining section, for example, at least one retaining element provided at the outflow end section of a heart valve prosthesis comprising a heart valve stent 50 and a prosthetic heart valve 60 not shown in FIG. 9a when the stent 50 is accommodated in the catheter tip 80-1. Conceivable here would be to configure the retaining means 74 of the stent holder 70 such that they can releasably engage with the at least one retaining element of stent 50.

In FIG. 9a, the retaining means 74 of stent holder 70 are for example configured as projecting elements which can be brought into engagement with retaining elements of a stent 50 configured correspondingly complementary thereto, for example in the form of eyelets. It would however also be conceivable for the retaining means 74 of stent holder 70 to be configured as cavities or recesses introduced into the cylindrical body of the stent holder 70 and designed to receive correspondingly complementary configured retaining elements of the heart valve stent 50.

With the catheter tip 80-1 for a medical delivery system designed for a transapical approach shown as an example in FIG. 9a, the stent holder 70 is preferably arranged to be stationary relative the (not shown) handle of the medical delivery system such that upon a rotation of the handle about the longitudinal axis of the medical delivery system, for example, the stent holder 70 will also be engaged in the rotational motion. It is hereby conceivable for the stent holder 70 to be connected to the handle via connecting means fixedly attached to the body of the handle.

On the other hand, the first receiving area 111 of the catheter tip 80-1 may preferably also movable in the longitudinal direction of the catheter tip 80-1 relative the stent holder 70 by means of appropriately manipulating a force transfer means. With the catheter tip 80-1 shown for example in FIG. 9a, an inner catheter 130, configured as a cannula tube extending from a distal end section of a handle (not shown in FIG. 9a) to the distal-side end tip 125 of the catheter tip 80-1 is employed as the force transfer means.

As indicated above, it is provided in the case of the catheter tip 80-1 for a medical delivery system designed for a transapical approach for the stent holder 70 of the catheter tip 80-1 to preferably be fixedly connected to a handle, a body of the handle respectively, so as to in particular freeze the freedom of rotational motion about the longitudinal axis of the medical delivery system respective the stent holder 70 as well as the freedom of motion in the direction of the longitudinal axis of the medical delivery system. Accordingly, the stent holder 70 is restricted from moving at least in the longitudinal direction of the medical delivery system relative the body of the handle. Rotational motion of the stent holder 70 about the longitudinal axis relative the handle is likewise eliminated.

It is to be emphasized that the inventive system for loading a stent, as needed with a prosthetic heart valve affixed thereto, into the tip of a catheter of a medical delivery system as disclosed herein is not only applicable to a catheter tip 80-1 for a medical delivery system designed for a transapical approach. In fact, it is equally possible to also use the system to load a stent system into a catheter tip of a medical delivery system designed for a transfemoral/transarterial approach.

The following, referencing FIG. 9b, will describe the design of an exemplary embodiment of a catheter tip 80-2 of a medical delivery system designed to transfemorally/transarterially introduce an expandable stent 50 into the body of a patient. The following description serves to present an example of a catheter tip 80-2 of a medical delivery system designed for a transfemoral/transarterial approach, whereby the inventive system and method can be employed to load a stent 50, as needed with a prosthetic heart valve 60 affixed thereto, into said catheter tip 80-2.

The catheter tip 80-2 depicted in FIG. 9b is part of a medical delivery system (not further shown) applicable for transfemorally/transarterially approaching a heart valve to be treated such as an aortic valve, for example. The medical delivery system enables an expandable heart valve stent 50 to be implanted into the body of a patient transfemorally or transartially, i.e. from the aortic arch. To this end, the delivery system comprises a catheter system (not shown in FIG. 9b), by means of which the heart valve stent 50 (likewise not shown in FIG. 9b) can be introduced into the body of the patient in its folded state.

The embodiment of the medical delivery system suited for a transarterial or transfemoral approach differs from the delivery system designed for transapical approach as described above referencing the FIG. 9a representation by the catheter tip 80-2 exhibiting a modified design to allow the transarterial approach to the site of implantation.

With regard to the design of the catheter tip 80-2 allowing the transarterial or transfemoral approach for the stent 50 accommodated in the catheter tip 80-2 to the site of implantation, it can be seen from FIG. 9b that the catheter tip 80-2—just like the catheter tip 80-1 of the delivery system designed for a transapical approach—comprises a stent holder 70 for releasably fixing for example the outflow end section of a heart valve prosthesis which can be accommodated in the catheter tip 80-2. Compared to the catheter tip 80-1 for the delivery system designed for a transapical approach, the retaining means 74 of the stent holder 70 configured as a crown are here provided at the distal end of the stent holder 70.

Furthermore, the catheter tip 80-2 of the delivery system designed for a transarterial/transfemoral approach comprises receiving means to receive a heart valve stent 50 with the prosthetic heart valve affixed thereto as needed. Specifically, the receiving means of the catheter tip 80-2 may consist of a first receiving area 111 to receive the distal end section of a stent 50, in particular the positioning holder of a stent 50, and a second receiving area 128 to receive the proximal end section of the stent 50, in particular the retaining arches of the stent 50 with the prosthetic heart valve 60 affixed thereto as needed.

As distinguished from the catheter tip 80-1 of the medical delivery system designed for a transapical approach as described with reference to FIG. 9a, in the catheter tip 80-2 of the medical delivery system designed for a transarterial/transfemoral approach pursuant FIG. 9b, the second receiving area 128 (stent funnel) serving to receive the proximal end section of the stent 50, and in particular the retaining arches of the stent 50 with the prosthetic heart valve 60 affixed as needed thereto, is arranged on the distal end section 125 of the catheter tip 80-2 while the first receiving area 111 (stent sleeve) is arranged between the second receiving area 128 and a handle (not shown in FIG. 9b).

In the catheter tip 80-2 of the medical delivery system as depicted in FIG. 9b designed for the transarterial approach to an insufficient or stenosed native heart valve, it is preferable to configure force transfer means, which connect actuating means of the handle to the second receiving area 128 (stent funnel) of the catheter tip 80-2, as an inner catheter 131 extending through the interior of an outer catheter or a sheath system. A further force transfer means which connects further actuating means of the handle to the first receiving area 111 (stent sleeve) of the catheter tip 80-2, is configured as an outer catheter, through the interior of which runs the other force transfer means configured as the inner catheter.

Upon the actuating of the associated actuating means, the second receiving area 128 (stent funnel) is movable in the longitudinal direction of the catheter tip 80-2 relative the stent holder 70 in the distal direction; i.e. away from the (not shown) handle, while the first receiving area 111 of catheter tip 80-2 is movable, upon the actuating of the correspondingly associated actuating means of the handle, in the longitudinal direction of the catheter tip 80-2 relative stent holder 70 in the proximal direction; i.e. toward the handle not shown in FIG. 9b.

The manipulations of the respective receiving areas 111, 128 of the catheter tip 80-2 of the delivery system designed for a transarterial/transfemoral approach effected by the actuating of the respective actuating means enables a sequential release of a stent 50 accommodated in the catheter tip 80-2, preferably at the site of implantation in the patient's heart.

According to an embodiment disclosed herein, a system 100 for loading a stent 50 onto a catheter tip 80, 80-1, 80-2 of a catheter system is provided. The catheter tip 80, 80-1, 80-2 is disposed at the distal end of the catheter system and is designed to accommodate the stent 50, said catheter tip 80, 80-1, 80-2 having retaining means 74 for releasably securing at least the outflow end of the heart valve prosthesis in the catheter tip 80, 80-1, 80-2.

In particular aspects of this embodiment, the catheter tip 80, 80-1, 80-2 has retaining means 74 shaped to co-operate with at least one retaining element 51 of the stent 50. In another particular aspect of the embodiment, the catheter tip 80, 80-1, 80-2 includes retaining means 74 having a crown 70 with at least one pocket 72, the at least one pocket 72 having a shape complementary to that of the at least one retaining element 51 of the stent 50.

In one particularly preferred embodiment, the stent 50 is part of a heart valve prosthesis, wherein the stent 50 has at least one retaining element 51 at the outflow end of the heart valve prosthesis. This at least one retaining element 51 can be engaged with corresponding retaining means 74 on an introduction catheter system, particularly a catheter tip or cartridge. In one embodiment of the at least one retaining element 51, the element 51 may be in the form of an anchoring eye disposed between two adjacent positioning arches of the stent. In which case, the arms of adjacent arches of the stent 50 are connected to the anchoring eye. It would likewise be conceivable for the arms of the adjacent arches to be directly connected to the at least one retaining element 51. On the other hand, the arms of the adjacent arches may also indirectly connected to the at least one retaining element 51 via a connecting web extending essentially in the longitudinal direction of the stent.

Generally speaking, the purpose of the at least one retaining element 51 provided on the outflow end of a heart valve prosthesis is to accommodate appropriate retaining means 74 on the introduction catheter system which complement that of the retaining element 51 of the stent 50. The engagement between the retaining means 74 of the catheter system on the one hand and the at least one retaining element 51 on the outflow end of the heart valve prosthesis on the other hand can be released by means of an external manipulation to release the stent 50 at the implantation site, thereby ensuring that the stent 50 expands and is thus reliably anchored. It will be appreciated that the at least one retaining element 51 of the stent 50 may be of any suitable shape or configuration such as eyes, loops, fingers or imperforate heads.

The use of such at least one retaining element 51 enables the stent 50 to remain in contact with the catheter prior to full release of the stent 50. By maintaining contact with the stent 50 prior to its full release, location and implantation position of the stent 50 can be controlled more accurately by a physician. The functioning of the stent 50 and heart valve prosthesis 60 attached to the stent 50 may also be checked and, if one or neither is functioning correctly, the physician can withdraw and remove the stent 50 by virtue of the at least one retaining element 51 of the stent 50 remaining in contact with the retaining means 74 of the catheter.

One embodiment of the retaining means 74 disposed in a catheter tip of an insertion or delivery system will be described in detail below with reference to FIGS. 10a to 10c.

FIG. 10a is a side view showing one embodiment of the retaining means 74. FIG. 10b is a view in cross-section along line A-A indicated in FIG. 10a, illustrating the embodiment of the retaining means 74, whilst FIG. 10c shows a plan view of an end region of an exemplary stent 50 where retaining elements 51 are provided. This end region of the stent 50 defines the outflow end of the heart valve prosthesis, when a prosthetic heart valve 60 is affixed to the stent 50. The retaining elements 51 of the stent 50 can be retained in a catheter tip of an insertion/delivery system by means of the retaining means 74 based on the embodiment illustrated in FIG. 10a.

As illustrated, the retaining means 74 provided at the catheter tip of the insertion/delivery system may have an essentially cylindrical body 70, the axis of symmetry of which lies on the longitudinal axis of the catheter tip. Several cut-outs or pockets 72—in FIG. 10b three in total—are spaced uniformly apart from one another in the material of the body 70 of the retaining means 74, preferably at the proximal end portion of the cylindrical body 70. These pockets 72 are connected to the proximal-end surface of the cylindrical body 70 by grooves 73.

The shape and size of the pockets 72 in the material of the body or crown 70 of the retaining means 74 are selected so that a retaining element 51 of the stent 50 complementing the pocket 72 can be accommodated, preferably positively, in each of the pockets 72. Thus, each retaining element 51 of the stent 50 establishes a releasable engagement with a pocket 72 formed in the crown 70 of the retaining means 74.

As illustrated in FIG. 10c, it is preferable in this respect if the retaining elements 51 of the stent 50 are provided in the form of projecting elements or projecting heads (retaining heads) at the end region of the stent 50. These retaining elements 51 of the stent 50 in the form of projecting elements may each connected to positioning arches 54 (and retaining arches 53) of the stent 50 via a neck portion or connecting web 56. When the retaining elements 51 of the stent 50 are positively held in the pockets 72 of the retaining means 74 of the catheter system, at least the ends of the neck portions 56 lie in the grooves 73.

Referring to FIG. 10b, the crown 70 of the retaining means 74 is cylindrical, wherein each of the pockets 72 formed in the crown 70 of the retaining means 74 has a shape adapted for substantially accommodating the retaining element 51 provided on the end region of the stent 50 such that there are no parts of the end region of the stent 50 protruding from the superficial surface of the cylindrical crown 70.

In addition, the crown 70 of the illustrated retaining means 74 may comprise snap-on means arranged on the at least one pocket 72 formed in the crown 70 of the retaining means 74 for releasable fixing the retaining element 51 provided on the end region of the stent 50 in the at least one pocket 72.

A valve prosthesis, i.e. a stent 50 with a prosthetic heart valve 60 affixed thereto, typically requires significant reduction of the expanded external dimension in order to be loaded onto a delivery device, such as a catheter tip 80, 80-1, 80-2 of a delivery system. In contrast, a typically coronary stent need only to be compressed a few millimeters in a size to reach its delivery configuration. Furthermore, it is necessary to avoid damaging the valve tissue of a valve prosthesis during the blooding procedure, wherein no such concern exists with a typical coronary stent. Accordingly, preferred embodiments of the present invention are well suited for use in loading an implantable prosthesis which includes a valve. However, the present loading system and methods may also be used in connection with, or adapted for use in connection with, non-valvular implants, such as coronary or other types of stents, for example.

FIG. 1 illustrates a system 100, which incorporates certain features, aspects, and advantages of the present invention. The system 100 is configured to facilitate compressing and releasably connecting a stent 50, in particular a self-expanding stent 50 having a replacement heart valve 60 affixed thereto, with retaining means 74 of a delivery catheter system, in particular with retaining means 74 provided in or at a catheter tip 80, 80-1, 80-2 of a delivery catheter system. In particular, the system 100 is configured to facilitate the loading of a valve prosthesis (cf. FIGS. 6a to 6g, FIGS. 7a to 7h and FIGS. 8a to 8j) onto a suitable, and preferably minimally invasive, delivery device, such as a catheter tip 80, 80-1, 80-2 of a delivery catheter of the kind as previously described with reference to FIGS. 9a and 9b for example.

Because the system 100 is especially well-suited for use in loading a prosthesis that incorporates a prosthetic heart valve, certain components of the system 100 are referred to herein using relative terminology to that components' relationship to the prosthetic heart valve. That is, certain components of the system 100 are named or otherwise described with respect to their position relative to an inflow end or an outflow end of the prosthetic heart valve of the implantable heart valve prosthesis. Generally, the components of the system 100 are referred to by which end of the heart valve prosthesis, either inflow or outflow, the component approaches the heart valve prosthesis during the illustrated loading procedure. However, the relative terminology used herein is employed as a matter of convenience for the reader and is not intended as a limitation on the present invention, unless specifically recited in the appended claims.

In addition, certain positions or directions of movement of components of the system 100 may be described in relation to movement relative to the catheter, in which a proximal end of the catheter is accessible external of the patient and is manipulated by a user of the system 100 and a distal end of the catheter is configured to support the valve prosthesis and is introduced into the patient.

The loading system 100 preferably includes fixing means 10 and centering means 30. In FIG. 1, one embodiment of the loading system 100 is shown in its assembled state, i.e. where the centering means 30 is connected to the fixing means 10. FIGS. 2a to 2c illustrate a preferred embodiment of the fixing means 10. FIGS. 3a to 3c illustrate a preferred embodiment of the centering means 30.

The fixing means 10 is configured to facilitate the fixation of a heart valve prosthesis to be loaded into a catheter tip 80, 80-1, 80-2. For this purpose, the fixing means 10 may comprise a cup-shaped element 11 having a rim zone 12 formed inside the cup-shaped element 11. By having such a rim zone 12 formed inside the cup-shaped element 11, a stent 50 or a heart valve prosthesis, i.e., a stent 50 with a prosthetic heart valve 60 affixed thereto, may be releasably connected to the fixing element 10.

In detail and as can be seen from for example FIG. 2a or FIG. 2c, the rim zone 12 formed inside the cup-shaped element 11 preferably has a side surface 12.1 extending substantially parallel to the longitudinal direction L of the cup-shaped element 11 and an upper surface 12.2 extending perpendicular thereto.

In one embodiment of the loading system 100, the fixing means 10 further comprises an annular element 12 having a substantially rectangular cross section. This annular element 12 is accommodated or can be received in the cup-shaped element 11 of the fixing element 10 such that the rim zone 12 is formed inside the cup-shaped element 11 for clamping a stent 50 or a heart valve prosthesis. In this embodiment, the inner diameter of the annular element 12 is preferably smaller than the outer diameter of the stent 50 or the heart valve prosthesis to be fixed with the fixing means 10.

As can be seen from FIG. 2c, an axially arranged cylindrical recessed portion 13 may be formed in the cup-shaped element 11 of the fixing means. This cylindrical recessed portion 13 defines an inner shell 13.1 and a bottom surface 13.2 of the cup-shaped element 11. For forming the rim zone 12 inside the cup-shaped element 11 which serves as clamping means for releasably fixing (clamping) a stent 50 or a heart valve prosthesis, in the embodiment depicted in FIG. 2c, an annular element 12 is accommodated in the recessed portion 13 and at least parts of same are connected to the inner shell 13.1 and the bottom surface 13.2.

In the bottom surface 13.2 of the cup-shaped element 11 a preferably circular opening 14 is provided. The diameter of this opening 14 is smaller than the inner diameter of the annular element 12. As will be described in more detail below, the opening 14 in the bottom surface 13.2 of the cup-shaped element 11 allows a catheter tip of a delivery system to pass through the loading system 100.

The cup-shaped element 11 and the annular element 12 may be separated pieces. In a preferred embodiment, however, the cup-shaped element 11 and the annular element 12 are integrally formed as one piece, preferably from a plastic material or any other suitable material, for example, metal material, such as stainless steel. However, other suitable materials may also be used, including polymeric materials or composites, for example.

The centering means 30 of the loading system 100 comprises a frustoconical housing 31 having an open end 31.2. In the assembled state of the loading system 100 (cf. FIG. 1), the open end 31.2 of the frustoconical housing 31 is opposite to the fixing means 10. The housing 31 is configured to compress a stent 50 or a heart valve prosthesis when the latter is moved through the housing 31. In detail and as can be seen in particular from for example FIGS. 3b and 3c, the housing 31 of the centering means 30 preferably comprises a first cylindrical end region 31.1 and an opposite second end region 31.2 which corresponds to the already mentioned open end 31.2 of the housing. Referring to FIG. 1, the first cylindrical end region 31.1 of the housing 31 is adapted such as to be at least partially receivable in the cup-shaped element 11 of the fixing means 10.

Desirably, a first opening provided in the first cylindrical end region 31.1 of the housing 31 is smaller than a second opening provided in the second cylindrical end region 31.2 of the housing 31 such that the surface of a through-hole 32 formed inside the housing 30 is tapered or moves closer to the axis L′ when moving along the surface from the second opening provided in the second cylindrical end region 31.2 to the first opening provided in the first cylindrical end region 31.1. Preferably, the surface of the trough-hole 32 is substantially linear in any plane passing through the axis L′. However, if desired, the surface may be nonlinear, such as a stepped or curved configuration, for example.

In a preferred embodiment of the centering means 30, the first cylindrical end region 31.1 of the housing 31 has a diameter which is larger than the inner diameter of the cup-shaped element 11 of the fixing means 10 at a region where the rim zone 12 is formed inside the cup-shaped element 11. for example, an annular element 12 having a substantially rectangular cross section may be accommodated in the cup-shaped element 11 such as to form the rim zone 12 inside the cup-shaped element 11, wherein the first cylindrical end region 31.1 of the housing 31 of the centering means 30 has a diameter which is larger than the inner diameter of the annular element 12.

In one embodiment of the loading system 100, the housing 31 of the centering means 30 preferably comprises a first cylindrical end region 31.1 connectable to the fixing means 10 and an opposite second end region 31.2, wherein an axially arranged frustoconical through-hole 32 is formed in the housing 31 of the centering means 30, said frustoconical through-hole 32 tapering toward the second end region 31.2 of said housing 31. In this embodiment, the diameter of the frustoconical through-hole 32 at the first end region 31.1 of the housing 31 is preferably smaller than the inner diameter of the cup-shaped element 11 at a region where the rim zone 12 is formed.

Also, it is possible that an annular element 12 having a substantially rectangular cross section is accommodated in the cup-shaped element 11 such as to form the rim zone 12 inside the cup-shaped element 11, wherein the diameter of the frustoconical through-hole 32 at the first end region 31.1 of the housing 31 of the centering means 30 is smaller than the inner diameter of the annular element 12.

The diameter of the frustoconical through-hole 32 at the second end region 31.2 of the housing 31 of the centering means 30 may be selected dependent on the diameter of the catheter tip 80 into which the stent 50 or heart valve prosthesis is to be loaded and/or dependent on the length of the stent 50 or heart valve prosthesis to be loaded.

As already mentioned above, the centering means 30 is releasable connectable with the fixing means 10. Preferably, the cup-shaped element 11 of the fixing means 10 is configured to be removably coupled to the housing 31 of the centering means 30. Any suitable means of connection between the cup-shaped element 11 and the housing 31 may be used. In the illustrated embodiment, an outer surface of the cup-shaped element 11 defines a generally J-shaped slot 16 (FIGS. 2a, 2c). Preferably, the cup-shaped element 11 includes multiple slots 16. In the illustrated embodiment, the cup-shaped element 11 includes three slots 16. Each slot 16 is configured to receive a projection 36 formed on an outside surface of the circumferential wall of the housing 31. Accordingly, the housing 31 of the centering means 30 may be secured to the cup-shaped element 11 of the fixing means 10 by twisting the housing 31 relative to the cup-shaped element 11. Other suitable means of connection may include corresponding threads or a frictional fit, for example, among other possibilities.

Reference is made to FIG. 4 which is a cross-sectional view of an exemplary embodiment of the loading system 100 in its unassembled state, i.e. a state in which the centering means 30 of the loading system 100 is not (yet) connected with the fixing means 10. In FIG. 4, a self-expanding stent 50 having a replacement heart valve 60 affixed thereto is connected to the fixing means 10 of the loading system 100.

Although the stent 50 as well as the prosthetic heart valve 60 affixed to the stent 50 are illustrated only in schematic fashion in FIG. 4, a valve prosthesis to be loaded into the catheter tip 80, 80-1, 80-2 of a medical delivery system preferably includes a frame or stent 50, which supports a prosthetic heart valve 60. The stent 50 preferably is an elongate, hollow structure comprised of a circumferential wall that preferably is of a framework or truss-type configuration made up of a plurality of strut portions. In one embodiment, the strut portions of the stent 50 are created by the removal of material between the strut portions, such as by laser cutting, for example. In other arrangement, the stent 50 may be constructed from a wire or collection of wires.

In certain preferred embodiments, the stent 50 is constructed from a shape memory material and may be collapsed or expanded in a cross-sectional dimension. It will be appreciated that the prosthetic heart valve 60 may be made from any suitable material, including biological valves removed from animals such as pigs and horses, man-made biological valves created from connective tissue such as pericardium, tissue grown from cell cultures, and man-made materials and fabrics such as Nitinol.

The prosthetic heart valve 60 has an inlet end and an outlet end. The outlet end preferably includes two or more cooperating valve leaflets. The inlet and outlet ends of the prosthetic heart valve 60 refer to a direction of blood flow through the prosthetic heart valve 60 when the prosthetic heart valve 60 is implanted within a patient. Thus, the heart valve prosthesis in general includes an inlet end and an outlet end, which refer to the direction of blood flow through the prosthesis/prosthetic heart valve 60 affixed to the stent 50.

As illustrated in FIG. 4, a heart valve prosthesis to be loaded into a catheter tip 80, 80-1, 80-2 of a delivery system is connected with the fixing means 10 of the loading system 100. In detail, the inflow end section of the heart valve prosthesis is clamped at the rim zone 12 formed inside the cup-shaped element 11 of the fixing means 10. For this purpose, the inflow end section of the heart valve prosthesis is slightly compressed and then inserted into the cup-shaped element 11. After releasing the heart valve prosthesis, the inflow end section radially expands and pushes against the side surface 12.1 of the rim zone 12 formed inside the cup-shaped element 11. In this way, the heart valve prosthesis is secured at the cup-shaped element 11 of the fixing means 10.

After connecting the heart valve prosthesis with the cup-shaped element 11 of the fixing means 10, the centering means 30 is moved in the direction of the arrow shown in FIG. 4. In detail, and as can be seen from FIG. 5, the centering means 30 is moved in the direction of the fixing means 10 such that the first end region 31.1 of the housing 31 is received inside the cup-shaped element 11. More precisely, the first end region 31.1 of the housing 31 abuts in its assembled state against the upper surface 12.2 of the rim zone 12 formed inside the cup-shaped element 11. When moving the centering means 30 in the direction of the cup-shaped element 11, an external dimension of at least a portion of the heart valve prosthesis is reduced as the heart valve prosthesis is moved into the frustoconical through-hole 32 formed in the housing 31 of the centering means 30. At the same time, the longitudinal axis of the heart valve prosthesis is generally aligned with the longitudinal axis L′ of the centering means 30 as well as the longitudinal axis L of the fixing means 10.

Preferably, the heart valve prosthesis is advanced within the frustoconical through-hole 32 formed in the housing 31 until retaining elements 51 of the stent 50 belonging to the heart valve prosthesis protrude from the through-hole opening at the second end region 32.2 of the housing 31. Accordingly, the inner surface of the frustoconical through-hole 32 operates to reduce an external dimension of at least a portion of the heart valve prosthesis as the heart valve prosthesis is moved relative to this surface.

With reference to FIG. 5, desirably the cup-shaped element 11 of the fixing means 10 as well as the housing 31 of the centering means 30 is utilized to advance the prosthesis into a desired final position within the frustoconical through-hole 32.

The cup-shaped element 11 is secured to the housing 31 of the centering means 30 and assist in maintaining a desired position of the heart valve prosthesis within the frustoconical through-hole 32.

As can be seem from FIG. 5, the diameter of the frustoconical through-hole 32 at the first end region 31.1 of the housing 31 of the centering means 30 is selected such that—when the centering means 30 is connected to the fixing means 10—an annular groove 15 is formed to receive parts of the stent 50 and, in particular, parts of the replacement heart valve 60 affixed to the stent 50. The annular groove 15 is at least partly limited by the side surface 12.1 of the rim zone 12 formed in the cup-shaped element 11, the bottom surface 13.2 of the cup-shaped element 11 and a base 33 of the first cylindrical end region 31.1 of the housing 31.

An exemplary method of loading a valve prosthesis onto the delivery catheter using the loading system 100 is described with reference to FIGS. 6a to 6g. Preferably, some or all of the steps illustrated and described with respect to FIGS. 6a to 6g are performed in an atmosphere that is within a range such that the frame of the valve prosthesis is in a martensite phase. Those of skill in the art will be able to determine an appropriate temperature range in which to perform the loading of other types of prostheses. In many instances, the atmosphere will be below room temperature. Desirably, all of the steps described below are performed in a cold fluid bath wherein the fluid is at or near a temperature of between about 2° C. and 8° C. In one arrangement, the cold fluid includes water which may contain other substances, e.g., a saline solution. A saline solution, is preferred because it is readily available at locations in which loading of the valve prosthesis may occur.

The procedure for loading a heart valve stent 50 having a heart valve prosthesis 60 affixed thereto into a catheter tip, for example a catheter tip 80-2 depicted in FIG. 9b, may correspond to the following method described hereinafter:

For loading a stent 50 or a stent 50 with a prosthetic heart valve 60 affixed thereto into a medical delivery system, in particular into a catheter tip 80 of a medical delivery system, the method may comprise the following method steps (cf. FIG. 6a):
furnishing a loading system 100 in accordance with anyone of the previously described exemplary embodiments of the present invention;
inserting a stent 50 or a stent 50 with a prosthetic heart valve 60 affixed thereto into the cup-shaped element 11 of the fixing means 10 of the loading system 100 such that the inflow end section of the heart valve prosthesis is clamped in the rim zone 12 formed inside the cup-shaped element 11;
inserting the catheter tip 80 of the medical delivery system through the opening 14 provided in the bottom surface of the cup-shaped element 11 and also through the stent 50 and the heart valve prosthesis 60 affixed to the stent 50 such that the retaining means 70 of the catheter tip 80 of the delivery catheter system is in the height of the retaining elements 51 formed at the outflow end section of the heart valve prosthesis; and
moving the centering means 30 of the loading system 10 relative to the catheter tip 80 and the fixing means 10 such as the catheter tip 80 passes through the through-hole 32 provided in the housing 31 of the centering means.

Thereafter, the centering means 30 is further advanced in the direction of the arrow depicted in FIG. 6a. In this regard, at least the inflow end section of the heart valve prosthesis clamped by the fixing means 10 is reduced in its diameter. At the same time, the retaining elements 51 provided at the outflow end section of the heart valve prosthesis engage with the retaining means 74 of the catheter tip 80 (cf. FIG. 6b).

Thereafter, the first receiving area 111 of the catheter tip 80, which is configured as a stent sheath, for example in the form of an elongated tube, is moved relative to the retaining means 74 in the direction shown by the arrow in FIG. 6b. Then, the retaining elements 51 of the heart valve prosthesis are overlapped by the proximal end section of the stent sheath 111. In other words, the proximal end section of the sleeve-type housing portion 111 of the catheter tip 80 is covering the retaining means 74. Accordingly, the inflow end section of the heart valve prosthesis is fixed to the retaining means 74 (cf. FIG. 6c).

Thereafter, the fixing means 10 of the loading system 100 is released from the centering means 30 (cf. FIG. 6d).

Then, the heart valve prosthesis is pushed through the through-hole 32 provided in the housing 31 of the centering means 30 thereby reducing the diameter of the heart valve prosthesis. At the same time, the sleeve-shaped element 111 of the catheter tip is moved in the proximal direction in order to cover all parts of the already reduced heart valve prosthesis (cf. FIG. 6e).

As an alternative, the centering means 30 could also be removed from the catheter tip 80 by moving the centering means 30 in the direction of the distal end tip 125 of catheter tip 80. Then, the compression of the heart valve prosthesis shall be performed manually.

Finally, the second sleeve-shaped element 128 of the catheter tip 80 may be moved relatively to the retaining means 74 in the distal direction as indicted by the arrow in FIG. 6f. Thereafter, the catheter tip is in its closed state and the heart valve prosthesis is properly secured and loaded in the catheter tip 80 (cf. FIG. 6g).

An alternative method of loading a valve prosthesis onto the delivery catheter using the loading system 100 is described with reference to FIGS. 7a to 7h. Again, some or all of the steps illustrated and described with respect to FIGS. 7a to 7h are performed in an atmosphere that is within a range such that the frame of the valve prosthesis is in a martensite phase. In many instances, the atmosphere will be below room temperature. Desirably, all of the steps described below are performed in a cold fluid bath wherein the fluid is at or near a temperature of between about 2° C. and 8° C. In one arrangement, the cold fluid includes water which may contain other substances, e.g., a saline solution. As already mentioned, a saline solution, is preferred because it is readily available at locations in which loading of the valve prosthesis may occur.

The procedure for loading a heart valve stent 50 having a heart valve prosthesis 60 affixed thereto into a catheter tip, for example a catheter tip 80-2 depicted in FIG. 9b, may correspond to the following method described hereinafter:

For loading a stent 50 or a stent 50 with a prosthetic heart valve 60 affixed thereto into a medical delivery system, in particular into a catheter tip 80 of a medical delivery system, the method may comprise the following method steps (cf. FIG. 7a):
furnishing a loading system 100 in accordance with anyone of the previously described exemplary embodiments of the present invention;
inserting the catheter tip 80 of the medical delivery system through the inflow end section of the stent 50 with the prosthetic heart valve 60 affixed thereto and also through the outflow end section of the stent 50 such that the catheter tip 80 extends along the blood flow passage provided in the stent 50 with the prosthetic heart valve 60 affixed thereto and at least partially beyond the outflow end section of the stent 50; and
inserting the catheter tip 80 of the medical delivery system through the opening 14 provided in the bottom surface of the cup-shaped element 11 and also through the frustoconical through-hole 32 formed in the centering means 30 of the loading system 100.

Thereafter, the inflow end section of the stent 50 is preferably manually compressed and at least the inflow end section of the stent 50 with the prosthetic heart valve 60 affixed thereto is inserted through the opening 14 provided in the bottom surface of the cup-shaped element 11 and also at least partly through the frustoconical through-hole 32 formed in the centering means 30 of the loading system 100 (cf. FIG. 7b).

Thereafter, the stent 50 with the prosthetic heart valve 60 affixed thereto is further advanced in the direction of the arrow depicted in FIG. 7b. In this regard, at least the inflow end section of the heart valve prosthesis clamped by the fixing means 10 is reduced in its diameter. At the same time, the retaining elements 51 provided at the outflow end section of the heart valve prosthesis engage with the retaining means 74 of the catheter tip 80 (cf. FIG. 7c).

Thereafter, the first receiving area 111 of the catheter tip 80, which is configured as a stent sheath, for example in the form of an elongated tube, is moved relative to the retaining means 74 in the direction shown by the arrow in FIG. 7c. Then, the retaining elements 51 of the heart valve prosthesis are overlapped by the proximal end section of the stent sheath 111. In other words, the proximal end section of the sleeve-type housing portion 111 of the catheter tip 80 is covering the retaining means 74. Accordingly, the inflow end section of the heart valve prosthesis is fixed to the retaining means 74 (cf. FIG. 7d).

Thereafter, the centering means 30 of the loading system 100 is released from the fixing means 10 and the released centering means 30 is removed from the catheter tip 80. At the same time, the fixing means 10 of the loading system 100 is released from the stent 50 with the prosthetic heart valve 60 affixed thereto and moved in the proximal direction, i.e. the direction to the handle of the catheter system (cf. FIG. 7e). The inflow end section of the heart valve prosthesis is still fixed to the retaining means 74 of the catheter tip 80.

Then, the heart valve prosthesis is manually compressed thereby reducing the diameter of the heart valve prosthesis. At the same time, the sleeve-shaped element 111 of the catheter tip is moved in the proximal direction in order to cover all parts of the already reduced heart valve prosthesis (cf. FIG. 7f).

Finally, the second sleeve-shaped element 128 of the catheter tip 80 may be moved relatively to the retaining means 74 in the distal direction as indicted by the arrow in FIG. 7g. Thereafter, the catheter tip is in its closed state and the heart valve prosthesis is properly secured and loaded in the catheter tip 80 (cf. FIG. 7h). Then, the fixing means 10 of the loading system 100 may be removed from the catheter tip 80.

Yet another alternative method of loading a valve prosthesis onto the delivery catheter using the loading system 100 is described with reference to FIGS. 8a to 8j. Again, some or all of the steps illustrated and described with respect to FIGS. 8a to 8j are performed in an atmosphere that is within a range such that the frame of the valve prosthesis is in a martensite phase. In many instances, the atmosphere will be below room temperature. Desirably, all of the steps described below are performed in a cold fluid bath wherein the fluid is at or near a temperature of between about 2° C. and 8° C. In one arrangement, the cold fluid includes water which may contain other substances, e.g., a saline solution. As already mentioned, a saline solution, is preferred because it is readily available at locations in which loading of the valve prosthesis may occur.

The procedure for loading a heart valve stent 50 having a heart valve prosthesis 60 affixed thereto into a catheter tip, for example a catheter tip 80-2 depicted in FIG. 9b, may correspond to the following method described hereinafter:

For loading a stent 50 or a stent 50 with a prosthetic heart valve 60 affixed thereto into a medical delivery system, in particular into a catheter tip 80 of a medical delivery system, the method may comprise the following method steps:
furnishing a loading system 100 in accordance with anyone of the previously described exemplary embodiments of the present invention, wherein the loading system 100 is in its assembled state, i.e. the cup-shaped element 11 of the fixing means 10 is secured to the housing 31 of the centering means 30 (cf. FIG. 8a);
furnishing a stent 50 or a stent 50 with a prosthetic heart valve 60 affixed thereto (cf. FIG. 8a); and
compressing, preferably manually compressing the inflow end section of the stent 50 to be loaded into the catheter tip 80 and inserting at least the inflow end section of the stent 50 with the prosthetic heart valve 60 affixed thereto through the opening 14 provided in the bottom surface of the cup-shaped element 11 and also at least partly through the frustoconical through-hole 32 formed in the centering means 30 of the loading system 100 (cf. FIG. 8b).

Thereafter, the stent 50 with the prosthetic heart valve 60 affixed thereto is further advanced in the direction of the arrow depicted in FIG. 8b. In this regard, at least the inflow end section of the heart valve prosthesis clamped by the fixing means 10 is reduced in its diameter (cf. FIG. 8c).

Then, the catheter tip 80 of the medical delivery system is inserted through the catheter tip 80 of the medical delivery system through the opening 14 provided in the bottom surface of the cup-shaped element 11 and also through the frustoconical through-hole 32 formed in the centering means 30 of the loading system 100. At the same time, the catheter tip 80 is also inserted through the inflow end section of the stent 50 with the prosthetic heart valve 60 affixed thereto and also through the outflow end section of the stent 50 such that the catheter tip 80 extends along the blood flow passage provided in the stent 50 with the prosthetic heart valve 60 affixed thereto and at least partially beyond the outflow end section of the stent 50 (cf. FIG. 8d).

Thereafter, the loading system 100 with the stent 50 and the prosthetic heart valve 60 affixed thereto is further advanced in the direction of the arrow depicted in FIG. 8d. In this regard, the retaining elements 51 provided at the outflow end section of the heart valve prosthesis engage with the retaining means 74 of the catheter tip 80 (cf. FIG. 8e).

Thereafter, the first receiving area 111 of the catheter tip 80, which is configured as a stent sheath, for example in the form of an elongated tube, is moved relative to the retaining means 74 in the direction shown by the arrow in FIG. 8e. Then, the retaining elements 51 of the heart valve prosthesis are overlapped by the proximal end section of the stent sheath 111. In other words, the proximal end section of the sleeve-type housing portion 111 of the catheter tip 80 is covering the retaining means 74. Accordingly, the inflow end section of the heart valve prosthesis is fixed to the retaining means 74 (cf. FIG. 8f).

Thereafter, the centering means 30 of the loading system 100 is released from the fixing means 10 and the released centering means 30 is removed from the catheter tip 80. At the same time, the fixing means 10 of the loading system 100 is released from the stent 50 with the prosthetic heart valve 60 affixed thereto and moved in the proximal direction, i.e. the direction to the handle of the catheter system (cf. FIG. 8g). The inflow end section of the heart valve prosthesis is still fixed to the retaining means 74 of the catheter tip 80.

Then, the heart valve prosthesis is manually compressed thereby reducing the diameter of the heart valve prosthesis. At the same time, the sleeve-shaped element 111 of the catheter tip is moved in the proximal direction in order to cover all parts of the already reduced heart valve prosthesis (cf. FIG. 8h).

Finally, the second sleeve-shaped element 128 of the catheter tip 80 may be moved relatively to the retaining means 74 in the distal direction as indicted by the arrow in FIG. 8i. Thereafter, the catheter tip is in its closed state and the heart valve prosthesis is properly secured and loaded in the catheter tip 80 (cf. FIG. 8j). Then, the fixing means 10 of the loading system 100 may be removed from the catheter tip 80.

Although the loading system 100 is advantageously configured for facilitating the loading of such a heart valve prosthesis, i.e. a stent 50 having a prosthetic heart valve 60 affixed thereon, it will be appreciated that the system 100 may be useful with other types of implants or prosthetics as well, including prosthetics that do or do not include a prosthetic heart valve 60.

The disclosed solution is not limited to the embodiments described with reference to the accompanying drawings. Also just as conceivable in fact are combinations of the individual features as specifically described.

1. A system for loading a stent into a catheter wherein the system comprises:
a first component for receiving a first portion of the stent; and
a second component for receiving a second portion of the stent, the second component being connectable to the first component;
wherein the first component comprises a cup-shaped element including a rim zone, the cup-shaped element having a longitudinal axis;
wherein the rim zone includes a side surface extending substantially parallel to the longitudinal axis of the cup-shaped element for receiving the first portion of the stent within the cup-shaped element; and
wherein the second component comprises a housing having an open end, the open end being opposite to the first component when the second component is connected to the first component, wherein the housing includes a frustoconical area configured to compress the stent when the stent is moved through the housing.

2. The system according to claim 1, wherein the first component comprises an annular element coupled to the cup-shaped element in the rim zone, the annular element including the side surface.

3. The system according to claim 2, wherein the annular element includes an upper surface substantially perpendicular to the longitudinal axis of the cup-shaped element.

4. The system according to claim 2, wherein the cup-shaped element includes an axially arranged cylindrical recessed portion having an inner shell and a bottom surface, and wherein the annular element is accommodated in the recessed portion such that at least a portion of the annular element contacts the inner shell and the bottom surface.

5. The system according to claim 4, wherein the bottom surface includes a circular opening having a diameter smaller than an inner diameter of the annular element.

6. The system according to claim 2, wherein the cup-shaped element and the annular element are integrally formed as one piece.

7. The system according to claim 1, wherein the housing of the second component comprises a first end region and a second end region opposite the first end region, wherein the first end region is cylindrical and at least partially receivable in the cup-shaped element of the first component.

8. The system according to claim 7, wherein the first end region has a diameter which is larger than an inner diameter of the cup-shaped element at a region in the rim zone.

9. The system according to claim 7, wherein the first component comprises an annular element coupled to the cup-shaped element in the rim zone, and wherein the first end region of the housing of the second component has a diameter which is larger than an inner diameter of the annular element.

10. The system according to claim 1, wherein the housing of the second component comprises a first end region connectable to the first component, the first end region being cylindrical, and a second end region opposite the first end region, and wherein the housing includes an axially arranged frustoconical through-hole tapering from the first end region toward the second end region.

11. The system according to claim 10, wherein a diameter of the frustoconical through-hole at the first end region of the housing is smaller than an inner diameter of the cup-shaped element at a region in the rim zone.

12. The system according to claim 10, wherein the first component comprises an annular element coupled to the cup-shaped element in the rim zone, and wherein a diameter of the frustoconical through-hole at the first end region of the housing of the second component is smaller than an inner diameter of the annular element.

13. The system according to claim 10, wherein a diameter of the frustoconical through-hole at the second end region of the housing of the second component is configured to receive a tip portion of the catheter.

14. The system according to claim 10, wherein when the second component is connected to the first component, an annular groove is formed inside the system to receive a portion of the stent that includes a replacement heart valve affixed to the stent, the annular groove being at least partly defined by the side surface of the rim zone of the cup-shaped element, a bottom surface of the cup-shaped element, and a base of the first end region of the housing.

15. The system according to claim 1, further comprising the stent, wherein a length of the housing of the second component is selected such that the stent (50) at least partially protrudes from the open end of the housing opposite to the first component.

16. The system according to claim 1, wherein the first component comprises at least one first locking element, and wherein the second component comprises at least one second locking element complementary to the first locking element of the first component to releasably lock the first component to the second component when the first and second components are connected in an assembled state of the system.

17. The system according to claim 16, wherein one of the first locking element of the first component or the second locking element of the second component is configured as an L-shaped slot, and the other of the first locking element or the second locking element is configured as a projecting pin receivable within the slot.

18-21. (canceled)

22. A system for loading a stent into a catheter, wherein the system comprises:
a first component for receiving a first portion of the stent, the first component including a recessed area defined by a side wall and a bottom surface, the bottom surface defining an opening; and
a second component for receiving a second portion of the stent, the second component including a lumen tapered from a lower end of the second component to an upper end of the second component, such that a diameter of the lumen at the lower end is greater than a diameter of the lumen at the upper end;
wherein the lower end of the second component is insertable into the recessed area of the first component; and
wherein the side wall includes a stepped portion, such that, when inserted into the first component, the lower end of the second component abuts the stepped portion to leave a space between the lower end of the second component and the bottom surface of the first component.

23. The system according to claim 22, wherein the first component includes at least one first locking element, and the second component includes at least one second locking element complementary to the first locking element for releasably locking the first component to the second component.

24. The system according to claim 22, wherein each of the first component and the second component has a generally cylindrical shape.

25. A system for loading a stent into a catheter, wherein the system comprises:
a first component for receiving a first portion of the stent, the first component including a recessed area defined by a side wall and a bottom surface, wherein the side wall includes a stepped portion and the bottom surface defines an opening; and
a second component for receiving a second portion of the stent, the second component including a lumen tapered from a lower end of the second component to an upper end of the second component, such that a diameter of the lumen at the lower end is greater than a diameter of the lumen at the upper end;
wherein the lower end of the second component is insertable into the recessed area of the first component; and
wherein a height of the recessed area is less than a height of the second component, such that, when the second component is fully inserted into the recessed area, the upper end of the second component extends above the first component.
